SEQUENCE LISTING (10 sequences)

SEQ ID NO: 1 - protein, 16 aa, Artificial Sequence
  LCDR1
RSSQSIVHSNGNTYLE

SEQ ID NO: 2 - protein, 7 aa, Artificial Sequence
  LCDR2
KVSNRFS

SEQ ID NO: 3 - protein, 9 aa, Artificial Sequence
  LCDR3
FQGSHVPLT

SEQ ID NO: 4 - protein, 5 aa, Artificial Sequence
  HCDR1
DYEMH

SEQ ID NO: 5 - protein, 17 aa, Artificial Sequence
  HCDR2
VIESETGGTAYNQKFKG

SEQ ID NO: 6 - protein, 16 aa, Artificial Sequence
  HCDR3
EGITTVATTYYWYFDV

SEQ ID NO: 7 - protein, 112 aa, Artificial Sequence [sequence omitted]

SEQ ID NO: 8 - protein, 125 aa, Artificial Sequence [sequence omitted]

SEQ ID NO: 9 - protein, 219 aa, Artificial Sequence
  LC
DVVMTQSPLSLPVTLGQPASISCRSSQSIVHSNGNTYLEWYLQKPGQSPQLLIYKVSNRFSGVPDRFSGSGSGTDFTLKISRVEAEDVGVYYCFQGSHVPLTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 10 - protein, 452 aa, Artificial Sequence
  HC
QGQLVQSGAEVKKPGASVKVSCKASGYTFTDYEMHWVRQAPIHGLEWIGVIESETGGTAYNQKFKGRVTITADKSTSTAYMELSSLRSEDTAVYYCAREGITTVATTYYWYFDVWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

(19) United States
(12) Patent Application Publication
(10) Pub. No.: US 2025/0034254 A1
(43) Pub. Date: Jan. 30, 2025

(54) USE OF ANTI-PD-1 ANTIBODY IN COMBINATION WITH CHEMOTHERAPY IN TREATING ESOPHAGEAL CANCER

(71) Applicant: SHANGHAI JUNSHI BIOSCIENCES CO., LTD., SHANGHAI (CN)

(72) Inventors: Sheng YAO, SHANGHAI (CN); Hui FENG, SHANGHAI (CN)

(73) Assignee: SHANGHAI JUNSHI BIOSCIENCES CO., LTD., SHANGHAI (CN)

(21) Appl. No.: 18/562,353

(22) PCT Filed: May 20, 2022
(86) PCT No.: PCT/CN2022/094057

§ 371 Date: Nov. 20, 2023

(30) Foreign Application Priority Data
May 20, 2021 (CN) 202110552089.2

(51) Int. Cl. C07K 16/28; A61K 31/337; A61K 33/243; A61K 39/00; A61P 35/04

(52) U.S. Cl. CPC C07K 16/2818; A61K 31/337; A61K 33/243; A61P 35/04; A61K 2039/505; A61K 2039/545; C07K 2317/565

(57) ABSTRACT

The present disclosure relates to a method of an anti-PD-1 antibody in combination with chemotherapy in treating esophageal cancer. In particular, the present disclosure relates to a method of a combination of an anti-PD-1 antibody or an antigen-binding fragment thereof and a chemotherapeutic agent in the preparation of a medicament for treating esophageal cancer. The present disclosure also relates to related drug combination and kit. The present disclosure also relates to a method of a reagent for detecting a gene amplification of chromosome 11q13 region in a test kit for predicting the therapeutic effect of the anti-PD-1 antibody and/or the antigen-binding fragment thereof on an esophageal cancer patient.

### FIELD

The present disclosure relates to use of an anti-PD-1 antibody in combination with chemotherapy in treating esophageal cancer. In particular, the present disclosure relates to a combination of an anti-PD-1 antibody or an antigen-binding fragment thereof, albumin-bound paclitaxel and cisplatin, and use thereof in the preparation of a medicament for treating advanced or metastatic esophageal cancer that has not previously been subjected to systemic chemotherapy. The present disclosure also relates to use of a reagent for detecting a gene amplification of chromosome 11q13 region in a test kit for predicting the therapeutic effect of the anti-PD-1 antibody and/or the antigen-binding fragment thereof on an esophageal cancer patient.

### BACKGROUND

Immune escape is one of the characteristics of cancer. It is disclosed that tumor-specific T lymphocytes are often present in the tumor microenvironment, draining lymph nodes and peripheral blood, but are generally unable to control tumor progression due to the network of immunosuppressive mechanisms present in the tumor microenvironment (see Ahmadzadeh, M. et al, Blood, 114: 1537-44). CD8⁺ tumor infiltrating T lymphocytes (TILs) generally express activation-induced inhibitory receptors, including CTLA-4 and PD-1, while tumor cells often express immunosuppressive ligands, including PD-1 ligand 1 (PD-L1, also called B7-H1 or CD274), which inhibits activation and effector functions of T cells. In the inhibitory mechanism, PD-1 and its ligands have become an important pathway for tumor cells to suppress activated T cells in the tumor microenvironment.

Programmed death receptor 1 (PD-1) plays an important role in immune regulation and maintenance of peripheral tolerance. PD-1 is expressed primarily in activated T and B cells and functions to suppress lymphocyte activation, which is a normal peripheral tissue tolerance mechanism of the immune system that prevents over-reactive immunity. However, the activated T cells infiltrated in the tumor microenvironment highly express PD-1 molecules, and inflammatory factors secreted by the activated leukocytes can induce the tumor cells to highly express ligands PD-L1 and PD-L2 of PD-1, resulting in the continuous activation of the PD-1 pathway of the activated T cells in the tumor microenvironment, and the suppression of T cells function to kill tumor cells. Therapeutic PD-1 antibodies can block this pathway, partially restore the function of T cells, and enable the activated T cells to continuously kill tumor cells.

Blocking the PD-1/PD-L1 pathway has proven to be an effective way to induce a durable anti-tumor response in various cancer indications over the last decade. Monoclonal antibodies (mAbs) blocking the PD/PD-L1 pathway can enhance activation and effector functions of tumor specific T cells, reduce tumor burden, and improve survival rate.

Esophageal cancer (EC) is one of the most common malignancies in humans, the morbidity and mortality of which has continued to rise over the past few decades, with 400,000 deaths worldwide per year now. Esophageal squamous cell carcinoma (ESCC) is the most common histological subtype of esophageal cancer in developing countries, and is the dominant histological subtype of esophageal cancer in South American and East Asian populations. The need for the treatment of this tumor remains unmet worldwide. The most commonly used chemotherapeutic agents for metastatic ESCC are cisplatin, 5-fluorouracil and taxanes. The 5-year survival rate of ESCC patients is low to 15%-20%. For advanced, recurrent and metastatic esophageal squamous carcinoma, paclitaxel and platinum-based combined chemotherapy is the current standard first-line treatment, while this treatment method has 40-60% of effective rate, 5.0 months of the median PFS and only 7-10 months of the median survival time, which cannot meet the clinical requirement.

### SUMMARY

The present disclosure provides use of a combination of an anti-PD-1 antibody or an antigen-binding fragment thereof and a chemotherapeutic agent in the preparation of a medicament for treating esophageal cancer.

In one or more embodiments, the esophageal cancer of the present disclosure is esophageal squamous cell carcinoma (ESCC), preferably advanced or metastatic esophageal squamous cell carcinoma, and more preferably advanced or metastatic esophageal squamous cell carcinoma that has not been subjected to systemic chemotherapy.

In one or more embodiments, the esophageal cancer of the present disclosure is esophageal cancer having a gene amplification of chromosome 11q13 region.

In one or more embodiments, the esophageal cancer of the present disclosure is esophageal cancer with a PD-L1 expression of <1% in immunohistochemical staining analysis of a tumor tissue section. In one or more embodiments, the esophageal cancer of the present disclosure is esophageal cancer with a PD-L1 expression of ≥1% in immunohistochemical staining analysis of a tumor tissue section. In one or more embodiments, the esophageal cancer of the present disclosure is esophageal cancer with a PD-L1 expression of <10% in immunohistochemical staining analysis of a tumor tissue section. In one or more embodiments, the esophageal cancer of the present disclosure is esophageal cancer with a PD-L1 expression of ≥10% in immunohistochemical staining analysis of a tumor tissue section.

In one or more embodiments, the esophageal cancer of the present disclosure is esophageal cancer with a tumor mutation burden (TMB) of less than 8 mutations/million base pairs; preferably, the esophageal cancer is esophageal cancer with a tumor mutation burden (TMB) of less than 6 mutations/million base pairs.

In one or more embodiments, the anti-PD-1 antibody or the antigen-binding fragment thereof of the present disclosure comprises a light chain complementarity determining region having amino acid sequences set forth in SEQ ID NOs: 1, 2 and 3, and a heavy chain complementarity determining region having amino acid sequences set forth in SEQ ID NOs: 4, 5 and 6.

In one or more embodiments, the anti-PD-1 antibody or the antigen-binding fragment thereof of the present disclosure comprises a light chain variable region having an amino acid sequence set forth in SEQ ID NO: 7 and a heavy chain variable region having an amino acid sequence set forth in SEQ ID NO: 8.

In one or more embodiments, the anti-PD-1 antibody of the present disclosure comprises a light chain having an amino acid sequence set forth in SEQ ID NO: 9 and a heavy chain having an amino acid sequence set forth in SEQ ID NO: 10.

In one or more embodiments, the anti-PD-1 antibody of the present disclosure is selected from one or more of nivolumab, pembrolizumab, toripalimab, Sintilimab, Camrelizumab, Tislelizumab and Cemiplimab, preferably toriplalimab.

In one or more embodiments, the chemotherapeutic agent of the present disclosure is selected from platinum and paclitaxel, preferably cisplatin and paclitaxel.

In one or more embodiments, the combination of the present disclosure is a combination of toripalimab, cisplatin and paclitaxel.

In one or more embodiments, in the use of the present disclosure,
the anti-PD-1 antibody or the antigen-binding fragment thereof is administered at a single dose of about 0.1 mg/kg body weight to about 10.0 mg/kg body weight, e.g., about 0.1 mg/kg body weight, 0.3 mg/kg body weight, 1 mg/kg body weight, 2 mg/kg body weight, 3 mg/kg body weight, 5 mg/kg body weight or 10 mg/kg body weight, or selected from a fixed dose of about 120 mg to about 480 mg, e.g., a fixed dose of about 120 mg, 240 mg, 360 mg or 480 mg, preferably a fixed dose of about 240 mg and about 360 mg;
the paclitaxel is administered at a single dose of about 130 mg/m² body surface area to about 230 mg/m² body surface area, e.g., about 130 mg/m² body surface area, 145 mg/m² body surface area, 160 mg/m² body surface area, 175 mg/m² body surface area, 190 mg/m² body surface area, 205 mg/m² body surface area or 230 mg/m² body surface area; and
the cisplatin is administered at a single dose of about 60 mg/m² body surface area to about 90 mg/m² body surface area, e.g., about 60 mg/m² body surface area, 65 mg/m² body surface area, 70 mg/m² body surface area, 75 mg/m² body surface area, 80 mg/m² body surface area, 85 mg/m² body surface area or 90 mg/m² body surface area.

In one or more embodiments, in the use of the present disclosure,
the anti-PD-1 antibody or the antigen-binding fragment thereof is administered at a frequency of about once every week, once every two weeks, once every three weeks, once every four weeks or once a month, preferably once every three weeks;
the paclitaxel is administered at a frequency of about once every week, twice every three weeks, once every two weeks, once every three weeks, once every four weeks or once a month, preferably once every three weeks; and
the cisplatin is administered at a frequency of about once every week, twice every three weeks, once every two weeks, once every three weeks, once every four weeks or once a month, preferably once every three weeks.

In one or more embodiments, in the use of the present disclosure,
the anti-PD-1 antibody or the antigen-binding fragment thereof is administered at a fixed dose of 240 mg or 360 mg once every three weeks; the paclitaxel is administered at a single dose of about 175 mg/m² body surface area once every three weeks; and the cisplatin is administered at a single dose of about 75 mg/m² body surface area once every three weeks.

In one or more embodiments, in the use of the present disclosure, the anti-PD-1 antibody or the antigen-binding fragment thereof, the paclitaxel and the cisplatin are administered for one week, two weeks, three weeks, one month, two months, three months, four months, five months, half a year or longer; in one embodiment, the durations of treatment cycles are the same or different, and the intervals between treatment cycles are the same or different.

In one or more embodiments, in the use of the present disclosure, the anti-PD-1 antibody or the antigen-binding fragment thereof, the paclitaxel and the cisplatin are administered for 4-6 cycles with one cycle being 3 weeks.

In one or more embodiments, in the use of the present disclosure, the anti-PD-1 antibody or the antigen-binding fragment thereof, the paclitaxel and the cisplatin are administered parenterally, e.g., by intravenous infusion, in a liquid dosage form, e.g., an injection.

In yet another embodiment, the present disclosure provides a drug combination comprising an anti-PD-1 antibody or an antigen-binding fragment thereof, paclitaxel and cisplatin.

In one or more embodiments,
the anti-PD-1 antibody or the antigen-binding fragment thereof in the drug combination of the present disclosure comprises a light chain complementarity determining region having amino acid sequences set forth in SEQ ID NOs: 1, 2 and 3, and a heavy chain complementarity determining region having amino acid sequences set forth in SEQ ID NOs: 4, 5 and 6; preferably, the anti-PD-1 antibody or the antigen-binding fragment thereof comprises a light chain variable region having an amino acid sequence set forth in SEQ ID NO: 7 and a heavy chain variable region having an amino acid sequence set forth in SEQ ID NO: 8; preferably, the anti-PD-1 antibody comprises a light chain having an amino acid sequence set forth in SEQ ID NO: 9 and a heavy chain having an amino acid sequence set forth in SEQ ID NO: 10; and more preferably, the anti-PD-1 antibody is toripalimab.

In one or more embodiments, the anti-PD-1 antibody or the antigen-binding fragment thereof in the drug combination of the present disclosure has an amount sufficient to provide more than one administration in a single dose: about 0.1 mg/kg body weight to about 10.0 mg/kg body weight, e.g., about 0.1 mg/kg body weight, 0.3 mg/kg body weight, 1 mg/kg body weight, 2 mg/kg body weight, 3 mg/kg body weight, 5 mg/kg body weight or 10 mg/kg body weight, or a fixed dose of about 120 mg to about 480 mg, e.g., a fixed dose of about 120 mg, 240 mg, 360 mg or 480 mg, preferably a fixed dose of about 240 mg and about 360 mg.

In one or more embodiments, the paclitaxel in the drug combination of the present disclosure has an amount sufficient to provide more than one administration in a single dose: about 130 mg/m² body surface area to about 230 mg/m² body surface area, e.g., about 130 mg/m² body surface area, 145 mg/m² body surface area, 160 mg/m² body surface area, 175 mg/m² body surface area, 190 mg/m² body surface area, 205 mg/m² body surface area or 230 mg/m² body surface area.

In one or more embodiments, the cisplatin in the drug combination of the present disclosure has an amount sufficient to provide more than one administration in a single dose: about 60 mg/m² body surface area to about 90 mg/m² body surface area, e.g., about 60 mg/m² body surface area, 65 mg/m² body surface area, 70 mg/m² body surface area, 75 mg/m² body surface area, 80 mg/m² body surface area, 85 mg/m² body surface area or 90 mg/m² body surface area.

In one or more embodiments, the drug combination is for administration to a patient in 4-6 treatment cycles, and each treatment cycle is one week, two weeks, three weeks, one month, two months, three months, four months, five months, half a year or longer; in one embodiment, the durations of treatment cycles are the same or different, and the intervals between the treatment cycles are the same or different.

In one or more embodiments, the drug combination is for administration to a patient in 4-6 treatment cycles with each treatment cycle being three weeks and an administration frequency being once every three weeks; the anti-PD-1 antibody or the antigen-binding fragment thereof has an amount sufficient to provide a single dose of 240 mg or 360 mg; the paclitaxel has an amount sufficient to provide a single dose of about 175 mg/m² body surface area; and the cisplatin has an amount sufficient to provide a single dose of about 75 mg/m² body surface area.

In yet another embodiment, the present disclosure provides a method of preventing or treating esophageal cancer, comprising: administering to an individual in need thereof a therapeutically effective amount of the anti-PD-1 antibody or the antigen-binding fragment thereof described herein in combination with a chemotherapeutic agent, or the drug combination described herein.

In one or more embodiments, in the method for preventing or treating esophageal cancer of the present disclosure, the esophageal cancer is esophageal squamous cell carcinoma (ESCC), preferably advanced or metastatic esophageal squamous cell carcinoma, and more preferably advanced or metastatic esophageal squamous cell carcinoma that has not been subjected to systemic chemotherapy.

In one or more embodiments, in the method for preventing or treating esophageal cancer of the present disclosure, the esophageal cancer is esophageal cancer having a gene amplification of chromosome 11q13 region.

In one or more embodiments, in the method for preventing or treating esophageal cancer of the present disclosure, the esophageal cancer is esophageal cancer with a PD-L1 expression of <1% in immunohistochemical staining analysis of a tumor tissue section. In one or more embodiments, the esophageal cancer is esophageal cancer with a PD-L1 expression of ≥1% in immunohistochemical staining analysis of a tumor tissue section. In one or more embodiments, the esophageal cancer is esophageal cancer with a PD-L1 expression of <10% in immunohistochemical staining analysis of a tumor tissue section. In one or more embodiments, the esophageal cancer is esophageal cancer with a PD-L1 expression of ≥10% in immunohistochemical staining analysis of a tumor tissue section.

In one or more embodiments, in the method for preventing or treating esophageal cancer of the present disclosure, the esophageal cancer is esophageal cancer with a tumor mutation burden (TMB) of less than 8 mutations/million base pairs; preferably, the esophageal cancer is esophageal cancer with a tumor mutation burden (TMB) of less than 6 mutations/million base pairs.

In one or more embodiments, in the method for preventing or treating esophageal cancer of the present disclosure, the anti-PD-1 antibody or the antigen-binding fragment thereof comprises a light chain complementarity determining region having amino acid sequences set forth in SEQ ID NOs: 1, 2 and 3, and a heavy chain complementarity determining region having amino acid sequences set forth in SEQ ID NOs: 4, 5 and 6.

In one or more embodiments, in the method for preventing or treating esophageal cancer of the present disclosure, the anti-PD-1 antibody or the antigen-binding fragment thereof comprises a light chain variable region having an amino acid sequence set forth in SEQ ID NO: 7 and a heavy chain variable region having an amino acid sequence set forth in SEQ ID NO: 8.

In one or more embodiments, in the method for preventing or treating esophageal cancer of the present disclosure, the anti-PD-1 antibody comprises a light chain having an amino acid sequence set forth in SEQ ID NO: 9 and a heavy chain having an amino acid sequence set forth in SEQ ID NO: 10.

In one or more embodiments, in the method for preventing or treating esophageal cancer of the present disclosure, the anti-PD-1 antibody is selected from one or more of nivolumab, pembrolizumab, toripalimab, Sintilimab, Camrelizumab, Tislelizumab and Cemiplimab, preferably toriplalimab.

In one or more embodiments, in the method for preventing or treating esophageal cancer of the present disclosure, the chemotherapeutic agent is selected from platinum and paclitaxel, preferably cisplatin and paclitaxel.

In one or more embodiments, in the method for preventing or treating esophageal cancer of the present disclosure, the combination is a combination of toripalimab, cisplatin and paclitaxel.

In one or more embodiments, in the method for preventing or treating esophageal cancer of the present disclosure, the anti-PD-1 antibody or the antigen-binding fragment thereof is administered at a single dose of about 0.1 mg/kg body weight to about 10.0 mg/kg body weight, e.g., about 0.1 mg/kg body weight, 0.3 mg/kg body weight, 1 mg/kg body weight, 2 mg/kg body weight, 3 mg/kg body weight, 5 mg/kg body weight or 10 mg/kg body weight, or selected from a fixed dose of about 120 mg to about 480 mg, e.g., a fixed dose of about 120 mg, 240 mg, 360 mg or 480 mg, preferably a fixed dose of about 240 mg and about 360 mg.

In one or more embodiments, in the method for preventing or treating esophageal cancer of the present disclosure, the paclitaxel is administered at a single dose of about 130 mg/m² body surface area to about 230 mg/m² body surface area, e.g., about 130 mg/m² body surface area, 145 mg/m² body surface area, 160 mg/m² body surface area, 175 mg/m² body surface area, 190 mg/m² body surface area, 205 mg/m² body surface area or 230 mg/m² body surface area.

In one or more embodiments, in the method for preventing or treating esophageal cancer of the present disclosure, the cisplatin is administered at a single dose of about 60 mg/m² body surface area to about 90 mg/m² body surface area, e.g., about 60 mg/m² body surface area, 65 mg/m² body surface area, 70 mg/m² body surface area, 75 mg/m² body surface area, 80 mg/m² body surface area, 85 mg/m² body surface area or 90 mg/m² body surface area.

In one or more embodiments, in the method for preventing or treating esophageal cancer of the present disclosure, the anti-PD-1 antibody or the antigen-binding fragment thereof is administered at a frequency of about once every week, once every two weeks, once every three weeks, once every four weeks or once a month, preferably once every three weeks.

In one or more embodiments, in the method for preventing or treating esophageal cancer of the present disclosure, the paclitaxel is administered at a frequency of about once every week, twice every three weeks, once every two weeks, once every three weeks, once every four weeks or once a month, preferably once every three weeks.

In one or more embodiments, in the method for preventing or treating esophageal cancer of the present disclosure, the cisplatin is administered at a frequency of about once every week, twice every three weeks, once every two weeks, once every three weeks, once every four weeks or once a month, preferably once every three weeks.

In one or more embodiments, in the method for preventing or treating esophageal cancer of the present disclosure, the anti-PD-1 antibody or the antigen-binding fragment thereof is administered at a fixed dose of 240 mg or 360 mg once every three weeks; the paclitaxel is administered at a single dose of about 175 mg/m² body surface area once every three weeks; and the cisplatin is administered at a single dose of about 75 mg/m² body surface area once every three weeks.

In one or more embodiments, in the method for preventing or treating esophageal cancer of the present disclosure, the anti-PD-1 antibody or the antigen-binding fragment thereof, the paclitaxel and the cisplatin are administered for one week, two weeks, three weeks, one month, two months, three months, four months, five months, half a year or longer; in one embodiment, the durations of treatment cycles are the same or different, and the intervals between treatment cycles are the same or different.

In one or more embodiments, in the method for preventing or treating esophageal cancer of the present disclosure, the anti-PD-1 antibody or the antigen-binding fragment thereof, the paclitaxel and the cisplatin are administered for 4-6 cycles with one cycle being 3 weeks.

In one or more embodiments, in the method for preventing or treating esophageal cancer of the present disclosure, the anti-PD-1 antibody or the antigen-binding fragment thereof and the chemotherapeutic agent are administered parenterally, e.g., by intravenous infusion.

In yet another embodiment, the present disclosure provides use of the anti-PD-1 antibody or the antigen-binding fragment thereof and the chemotherapeutic agent as described herein, or the drug combination described herein in the treatment or prevention of esophageal cancer; preferably, the esophageal cancer is esophageal squamous cell carcinoma. Further preferably, the esophageal cancer is as described in any one of the embodiments herein.

In yet another embodiment, the present disclosure provides use of a reagent for detecting a gene amplification of chromosome 11q13 region in the preparation of a test kit for predicting the therapeutic effect of a combination of an anti-PD-1 antibody and/or an antigen-binding fragment thereof and a chemotherapeutic agent on an esophageal cancer patient; preferably, the esophageal cancer is esophageal squamous cell carcinoma. In one embodiment, the esophageal cancer is as described in any one of the embodiments herein. In one embodiment, the anti-PD-1 antibody and/or the antigen-binding fragment thereof and chemotherapeutic agent are as described in any one of the embodiments herein.

In yet another embodiment, the present disclosure provides a kit comprising:
one or more single dosage units of the anti-PD-1 antibody or the antigen-binding fragment thereof described herein, one or more single dosage units of paclitaxel and one or more single dosage units of cisplatin; or
one or more single dosage units of the drug combination described herein.

In one or more embodiments, the kit of the present disclosure comprises:
the anti-PD-1 antibody or the antigen-binding fragment thereof in one or more single dosage units comprising the anti-PD-1 antibody or the antigen-binding fragment thereof at a dose of about 120 mg to about 480 mg, e.g., a dose of 120 mg, 240 mg, 360 mg or 480 mg, preferably 240 mg or 360 mg; and
the paclitaxel in one or more single dosage units comprising the paclitaxel at about 130 mg/m² body surface area to about 230 mg/m² body surface area, e.g., about 130 mg/m² body surface area, 145 mg/m² body surface area, 160 mg/m² body surface area, 175 mg/m² body surface area, 190 mg/m² body surface area, 205 mg/m² body surface area or 230 mg/m² body surface area, preferably 175 mg/m² body surface area; and the cisplatin in one or more single dosage units comprising the cisplatin at about 60 mg/m² body surface area to about 90 mg/m² body surface area, e.g., about 60 mg/m² body surface area, 65 mg/m² body surface area, 70 mg/m² body surface area, 75 mg/m² body surface area, 80 mg/m² body surface area, 85 mg/m² body surface area or 90 mg/m² body surface area, preferably 75 mg/m² body surface area.

In one embodiment, the kit of the present disclosure is for use in the use or method according to any one of the embodiments herein.

### BRIEF DESCRIPTION OF THE DRAWINGS

FIG. 1 shows the overall survival (OS) Kaplan-Meier curve (intention to treat analysis, ITT) evaluated according to RECIST 1.1 criteria.

FIG. 2 shows a subgroup analysis (ITT) of overall survival (OS) evaluated according to RECIST 1.1 criteria.

FIG. 3a shows the PFS Kaplan-Meier curve (ITT) evaluated by the blind independent review committee (BIRC) according to RECIST 1.1 criteria.

FIG. 3b shows the PFS Kaplan-Meier curve (ITT) evaluated by researchers according to RECIST 1.1 criteria.

FIG. 4 shows the PFS subgroup analysis (ITT) evaluated by the blind independent review committee (BIRC) according to RECIST 1.1 criteria.

### DETAILED DESCRIPTION

The present disclosure relates to a method for treating esophageal cancer. The method of the present disclosure comprise administering to a patient in need thereof an anti-PD-1 antibody or an antigen-binding fragment thereof in combination with a chemotherapeutic agent.

### Terminology

In order to facilitate the understanding of the present disclosure, some terms are specifically defined below. Unless otherwise specifically defined herein, all terms used herein have the same meaning as commonly understood disclosure.

“Administering”, “giving” and “treating” refers to introducing a composition comprising a therapeutic agent into a subject using any one of a variety of methods or delivery systems. Routes of administration of the anti-PD-1 antibody include intravenous, intramuscular, subcutaneous, intraperitoneal, spinal or other parenteral routes of administration, such as injection or infusion. “Parenteral administration” refers to modes of administration apart from enteral or local administration, typically by injection, including but not limited to, intravenous, intramuscular, intra-arterial, intrathecal, intralymphatic, intralesional, intracapsular, intraorbital, intracardiac, intradermal, intraperitoneal, transtracheal, subcutaneous, subcuticular, intra-articular, subcapsular, subarachnoid, intraspinal, epidural and intrasternal injection and infusion, and in vivo electroporation.

An “adverse event” (AE) described herein is any adverse and often unintended or undesirable sign, symptom, or disease associated with the use of medical treatment. For example, an adverse event may be associated with the activation of the immune system or the expansion of immune system cells in response to treatment. The medical treatment may have one or more related AEs, and each AE may have the same or a different severity level.

“Tumor burden” refers to the total amount of tumor mass distributed throughout the body. Tumor burden refers to the total number of cancer cells or the total size of the tumor throughout the body. Tumor burden can be determined by a variety of methods known in the art, such as measuring the size of a tumor using calipers after the tumor is removed from a subject, or using imaging techniques (e.g., ultrasound, bone scanning, computed tomography (CT), or magnetic resonance imaging (MRI) scanning) when the tumor is in vivo.

The term “tumor size” refers to the total size of a tumor, which can be measured as the length and width of the tumor. Tumor size can be determined by a variety of methods known in the art, such as measuring the size of a tumor using calipers after the tumor is removed from a subject, or using imaging techniques (e.g., bone scanning, ultrasound, CT, or MRI scanning) when the tumor is in vivo.

The term “subject” and “individual” include any organism, preferably an animal, more preferably a mammal (such as rat, mouse, dog, cat and rabbit), and most preferably a human. The terms “subject” and “patient” are used interchangeably herein.

An “antibody” described herein refers to any form of antibody that achieves a desired bioactivity or binding activity. Therefore, it is used in the broadest sense, but is not limited to, monoclonal antibodies, polyclonal antibodies, multispecific antibodies, humanized full-length human antibodies, chimeric antibodies and camelized single-domain antibodies. The “antibody” specifically binds to an antigen and comprises at least two heavy (H) chains and two light (L) chains interconnected by disulfide bonds. Each heavy chain comprises a heavy chain variable region (VH) and a heavy chain constant region comprising three constant domains CH1, CH2 and CH3. Each light chain comprises a light chain variable region (VL) and a light chain constant region comprising one constant domain CL. The VH and VL regions can be further divided into hypervariable regions termed complementarity determining regions (CDRs), which are scattered over more conserved regions termed framework regions (FRs). Generally, both light and heavy chain variable domains comprise FR1, CDR1, FR2, CDR2, FR3, CDR3 and FR4 from N-terminus to C-terminus. Amino acids are typically assigned to each domain according to the following definitions: Sequences of Proteins of Immunological Interest, Kabat et al; National Institutes of Health, Bethesda, Md.; 5^{th} edition; NIH publication No. 91-3242 (1991): Kabat (1978) Adv. Prot. Chem. 32:1-75; Kabat et al, (1977) J. Biol. Chem. 252:6609-6616; Chothia et al, (1987) J Mol. Biol. 196:901-917 or Chothia et al, (1989) Nature 341:878-883.

The carboxyl-terminal portion of the heavy chain can define a constant region primarily responsible for effector function. Human light chains are generally classified as κ and λ chains. Human heavy chains are generally classified as μ, δ, γ, α or ε chains, and isotypes of the antibody are defined as IgM, IgD, IgG, IgA and IgE, respectively. IgG subclass includes, but is not limited to, IgG1, IgG2, IgG3 and IgG4.

The term “antibody” includes: naturally occurring and non-naturally occurring Abs; monoclonal and polyclonal Abs; chimeric and humanized Abs; human or non-human Abs; fully synthetic Abs; and single chain Abs. Non-human Abs can be humanized by recombinant methods to reduce their immunogenicity in humans.

Unless otherwise specifically indicated, an “antibody fragment” or “antigen-binding fragment” described herein refers to an antigen-binding fragment of an antibody, i.e., an antibody fragment that retains the ability of a full-length antibody to specifically bind to an antigen, e.g., a fragment that retains one or more CDR regions. Examples of antigen-binding fragment include, but are not limited to, Fab, Fab′, F(ab′)₂, and Fv fragments; a diabody; a linear antibody; a single-chain antibody molecule; and a nanoantibody and a multispecific antibody formed from fragments of the antibody.

A “chimeric antibody” refers to an antibody and a fragment thereof in which a portion of the heavy and/or light chain is identical with or homologous to corresponding sequences of an antibody derived from a particular species (e.g., human) or belonging to a particular antibody class or subclass, while the remainder of the chain is identical with or homologous to corresponding sequences of an antibody derived from another species (e.g., mouse) or belonging to another antibody class or subclass, so long as they exhibit the desired bioactivity.

A “human antibody” refers to an antibody that comprises only human immunoglobulin sequences. A human antibody may contain a murine carbohydrate chain if it is produced in mice, mouse cells, or hybridomas derived from mouse cells. Similarly, “mouse antibody” or “rat antibody” refers to an antibody that comprises only mouse or rat immunoglobulin sequences, respectively.

A “humanized antibody” refers to an antibody form containing sequences from both non-human (e.g., murine) and human antibodies. Such antibodies contain minimal sequences derived from non-human immunoglobulins. Typically, a humanized antibody will comprise substantially all of at least one and typically two variable domains, in which all or substantially all of the hypervariable loops correspond to those of a non-human immunoglobulin and all or substantially all of the FR regions are those of a human immunoglobulin. The humanized antibody in one embodiment also comprises at least one portion of an immunoglobulin constant region (Fc), typically a human immunoglobulin constant region.

The term “cancer” or “malignancy” used herein refers to a wide variety of diseases characterized by the uncontrolled growth of abnormal cells in the body. Unregulated cell division, growth division and growth lead to the formation of malignancies that invade adjacent tissues and can also metastasize to distal parts of the body through the lymphatic system or the blood flow. Examples of cancers suitable for treatment or prevention using the method, medicament and kit of the present disclosure include, but are not limited to, carcinoma, lymphoma, leukemia, blastoma and sarcoma. More specific examples of cancer include squamous cell cancer, myeloma, small-cell lung cancer, esophageal cancer, glioma, Hodgkin lymphoma, non-Hodgkin lymphoma, acute myeloid leukemia, multiple myeloma, gastrointestinal cancer, renal cancer, ovarian cancer, liver cancer, lymphoblastic leukemia, lymphocytic leukemia, colorectal cancer, endometrial cancer, kidney cancer, prostate cancer, thyroid cancer, melanoma, chondrosarcoma, neuroblastoma, pancreatic cancer, glioblastoma multiforme, nasopharyngeal cancer, cervical cancer, brain cancer, gastric cancer, bladder cancer, hepatoma, breast cancer, colon cancer, and head and neck cancer.

The term “tumor mutation burden (TMB)” herein refers to the total number of gene coding errors, base substitutions, gene insertion or deletion errors detected in a somatic cell per million bases. In some embodiments of the present disclosure, tumor mutation burden (TMB) is estimated by analysis of somatic mutations, including coding base substitutions and the megabase insertions of the panel sequences studied.

The term “esophageal cancer” or “EC” is one of the most common malignancies in humans. It is a common gastrointestinal tumor, and can be divided into squamous cell carcinoma, adenocarcinoma, and small cell carcinoma according to histological and pathological typing. Among them, “esophageal squamous cell carcinoma” or “esophageal squamous carcinoma” or “ESCC” is the most common type of EC in developing countries, and the prognosis for the treatment of this cancer is very poor with the 5-year overall survival rate of only 20%-30%.

The term “immunotherapy” refers to the treatment of a subject with a disease or at risk of infection or disease recurrence by a method that includes inducing, enhancing, suppressing or otherwise modifying an immune response. The “treatment” or “therapy” of a subject refers to any type of intervention or process performed on the subject, or the administration of an active agent to the subject, with the purpose of reversing, alleviating, ameliorating, slowing or preventing the onset, progression, severity, or recurrence of symptoms, complications or conditions, or biochemical indicators associated with the disease.

A “programmed death receptor-1 (PD-1)” refers to an immunosuppressive receptor belonging to the CD28 family. PD-1 is expressed primarily on previously activated T cells in vivo and binds to two ligands, PD-L1 and PD-L2. The term “PD-1” used herein includes human PD-1 (hPD-1), variants, isotypes, and species homologs of hPD-1, and analogs having at least one common epitope with hPD-1.

A “therapeutically effective amount” or “therapeutically effective dose” of a medicament or therapeutic agent is any amount of the medicament that, when used alone or in combination with an additional therapeutic agent, protects a subject from the onset of a disease or promotes the regression of a disease as evidenced by a decrease in the severity of disease symptoms, an increase in the frequency and duration of disease symptom-free phase, or the prevention of injury or disability resulting from the affliction of the disease. The ability of a therapeutic agent to promote the regression of a disease can be assessed using a variety of methods, such as in human subjects during clinical trials, in animal model systems that predict human efficacy, or by determining the activity of the agent in an in vitro assay.

A therapeutically effective amount of a medicament includes a “prophylactically effective amount” which is any amount of a medicament that, when administered alone or in combination with an anti-neoplastic agent, inhibits the development or recurrence of cancer to a subject at risk of developing cancer or a subject having cancer recurrence.

A “biotherapeutic agent” refers to a biological molecule, such as an antibody or fusion protein, which blocks ligand/receptor signaling in any biological pathway that supports tumor maintenance and/or growth or inhibits an anti-tumor immune response.

Unless otherwise specifically indicated, “CDR” used herein refers to a complementarity determining region of the immunoglobulin variable region defined using the Kabat numbering system.

A “therapeutic anti-PD-1 monoclonal antibody” refers to an antibody that specifically binds to the mature form of a specific PD-1 expressed on the surface of certain mammalian cells. Mature PD-1 does not have a secretory leader sequence, or leader peptide. The terms “PD-1” and “mature PD-1” are used interchangeably herein and are to be understood as the same molecule unless otherwise specifically defined, or clearly seen from the context.

A therapeutic anti-human PD-1 antibody or anti-hPD-1 antibody described herein refers to a monoclonal antibody that specifically binds to mature human PD-1.

A “framework region” or “FR” described herein refers to the immunoglobulin variable region excluding CDR regions.

An “isolated antibody or antigen-binding fragment thereof” refers to a molecule that is in a purified state, and in this case, is substantially free of other biomolecules, such as nucleic acids, proteins, lipids, carbohydrates, or other materials (such as cell debris or growth medium).

A “patient” or “subject” refers to any single subject in need of a medical procedure or participating in a clinical trial, epidemiological study, or serving as a control, and is generally a mammal, including human and other mammals, such as horses, cows, dogs or cats.

The “RECIST 1.1 efficacy criteria” described herein refers to the definition of target injury and non-target injury described in Eisenhauver et al, E. A. et al, Eur. J Cancer 45:228-247(2009) in the context of the measured background. RECIST 1.1 efficacy criteria is the most commonly used criteria for efficacy evaluation of solid tumors prior to immunotherapy. However, with the advent of the immunotherapy, many problems which have not been found in the previous tumor evaluation, so based on the new phenomenon brought by immunotherapy itself, RECIST working group provides a new determination standard after correcting the existing “RECIST v.1.1” in 2016, namely the “irRECIST standard” described herein, aiming at better evaluating the efficacy of immunotherapeutic drugs.

The term “ECOG” score standard is an indicator of general health status and tolerance to treatment of patients from their physical strength. ECOG score standard for the physical strength is 0 points, 1 point, 2 points, 3 points, 4 points and 5 points. A score of 0 means that the motility is completely normal and has no difference from the motility before onset of disease. A score of 1 means that the person is free to walk and engages in light physical activities, including general housework or office work, but not in heavy physical activities.

A “sustained response” refers to a sustained therapeutic effect following cessation of treatment with a therapeutic agent or combination therapy described herein. In some embodiments, the sustained response has a duration that is at least the same as the duration of treatment or at least 1.5, 2.0, 2.5 or 3 times the duration of the treatment.

A “tissue section” refers to a single portion or piece of a tissue sample, such as a tissue slice cut from a sample of normal tissue or a tumor.

As used herein, “treating” cancer refers to administering a treatment regimen described herein (e.g., administration of an anti-PD-1 antibody) to a subject with or diagnosed with cancer to achieve at least one positive therapeutic effect (e.g., a decrease in cancer cell number, a decrease in tumor volume, a reduction in the rate of cancer cell infiltration into peripheral organs, or a reduction in the rate of tumor metastasis or tumor growth). Positive therapeutic effects in cancer can be measured in a variety of ways (see W. A. Weber, J. Nucl. Med., 50:1S-10S (2009)). For example, T/C≤42% for tumor growth inhibition is the minimum level of anti-tumor activity according to the NCI criteria. It is considered that T/C (%)=median treated tumor volume/median control tumor volume×100. In some embodiments, the therapeutic effect achieved by the combination of the present disclosure is any one of PR, CR, OR, PFS, DFS and OS. PFS (also called “time to tumor progression”) refers to the length of time during and after treatment during which cancer does not grow, and includes the amount of time a patient experiences CR or PR and the amount of time a patient experiences SD. DFS refers to the length of time during and after treatment during which a patient remains disease-free. OS refers to an extension of life expectancy compared to an initial or untreated individual or a patient. In some embodiments, the response to the combination of the present disclosure is any one of PR, CR, PFS, DFS, OR OS, assessed using RECIST 1.1 efficacy criteria. The treatment regimen of the combination of the present disclosure effective in treating a cancer patient may vary depending upon a variety of factors such as the disease state, age, weight of the patient and the ability of the therapy to elicit an anti-cancer response in the subject. Embodiments of the present disclosure may not achieve an effective positive therapeutic effect in each subject, but should be effective and achieve a positive therapeutic effect in a statistically significant number of subjects.

The terms “mode of administration” and “dosing regimen” are used interchangeably and refer to the dosage and time of use of each therapeutic agent in the combination of the present disclosure.

The term “immunohistochemistry (IHC)” refers to a method for determining antigens (polypeptides and proteins) in tissue cells by developing chromogenic agents (fluoresceins, enzymes, metal ions, isotopes) that label antibodies through chemical reaction based on the principle that antigens specifically bind to antibodies, and performing localized, qualitative and relatively quantitative studies on those antigens. In some embodiments of the present disclosure, a tumor tissue sample from a subject is tested for PD-L1 prior to treatment with an anti-PD-1 antibody by staining the anti-human PD-L1 antibody SP142 from Roche (Cat No: M4422). In some embodiments, tumor cells with membrane staining ≥1% are defined as PD-L1 positive.

In the following paragraphs, various embodiments of the present disclosure are described in further detail.

### Anti-PD-1 Antibody

The “PD-1 antibody” herein refers to any chemical compound or biomolecule that binds to the PD-1 receptor, blocks the binding of PD-L1 expressed on cancer cells to PD-1 expressed on immune cells (T, B and NK cells), and preferably blocks the binding of PD-L2 expressed on cancer cells to PD-1 expressed on immune cells. Alternative nouns or synonyms for PD-1 and its ligands include: PDCD1, PD1, CD279 and SLEB2 for PD-1; PDCD1L1, PDL1, B7-H1, B7H1, B7-4, CD274 and B7-H for PD-L1; and PDCD1L2, PDL2, B7-DC and CD273 for PD-L2. In any of the therapy, medicament and use described herein for treating a human individual, the PD-1 antibody blocks the binding of human PD-L1 to human PD-1, and preferably blocks the binding of both human PD-L1 and PD-L2 to human PD1. The amino acid sequence of human PD-1 can be found at NCBI locus number: NP_005009. The amino acid sequences of human PD-L1 and PD-L2 can be found at NCBI locus numbers: NP 054862 and NP 079515.

As used herein, unless otherwise indicated or described, when referring to the term “anti-PD-1 antibody”, it includes antigen-binding fragments of the antibody.

The anti-PD-1 antibody suitable for any of the use, therapy, drug combination and kit of the present disclosure binds has an immunosuppressive effect achieved by binding to PD-1 with high specificity and high affinity, blocking the binding of PD-L1/2 to PD-1 and inhibiting PD-1 signal transduction. In any of the use, therapy, drug combination and kit disclosed herein, the anti-PD-1 antibody includes the full-length antibody itself, as well as an antigen-binding portion or fragment that binds to the PD-1 receptor and exhibits functional properties similar to an intact Ab in inhibiting ligand binding and upregulating the immune system. In some embodiments, the anti-PD-1 antibody or the antigen-binding fragment thereof is an anti-PD-1 antibody or an antigen-binding fragment thereof that cross-competes for binding to human PD-1 with toripalimab. In other embodiments, the anti-PD-1 antibody or the antigen-binding fragment thereof is a chimeric, humanized or human Ab or an antigen-binding fragment thereof. In some embodiments for treating a human individual, the Ab is a humanized Ab.

In some embodiments, the anti-PD-1 antibody that can be used in any of the use, therapy, drug combination and kit described herein includes a monoclonal antibody (mAb) or an antigen-binding fragment thereof that specifically binds to PD-1, and preferably specifically binds to human PD-1. The mAb can be a human antibody, a humanized antibody, or a chimeric antibody, and can include a human constant region. In some embodiments, the constant region is selected from human IgG1, IgG2, IgG3 and IgG4 constant regions; preferably, the anti-PD-1 antibody or the antigen-binding fragment thereof suitable for use in any of the use, therapy, drug combination and kit described herein comprises a heavy chain constant region of human IgG1 or IgG4 isotype, more preferably a human IgG4 constant region. In some embodiments, the sequence of the IgG4 heavy chain constant region of the anti-PD-1 antibody or the antigen-binding fragment thereof comprises the S228P mutation that replaces a serine residue in the hinge region with a proline residue that is typically present at the corresponding position of an antibody of IgG1 isotype.

In any one of the embodiments of the use, therapy, drug combination and kit of the present disclosure, the PD-1 antibody is a monoclonal antibody or an antigen-binding fragment thereof, and the light chain CDRs thereof comprise amino acid sequences set forth in SEQ ID NOs: 1, 2 and 3, and the heavy chain CDRs thereof comprise amino acid sequences set forth in SEQ ID NOs: 4, 5 and 6.

In any one of the embodiments of the use, therapy, drug combination and kit of the present disclosure, the PD-1 antibody is a monoclonal antibody that specifically binds to human PD-1 and comprises: (a) a light chain variable region set forth in SEQ ID NO: 7, and (b) a heavy chain variable region set forth in SEQ ID NO: 8.

In any one of the embodiments of the use, therapy, drug combination and kit of the present disclosure, the PD-1 antibody specifically binds to human PD-1 and comprises: (a) a light chain variable region set forth in SEQ ID NO: 9, and (b) a heavy chain set forth in SEQ ID NO: 10.

Table A below provides the amino acid sequence numbering of the light chain CDRs and heavy chain CDRs of an exemplary anti-PD-1 antibody mAb for use in the use, therapy, drug combination and kit of the present disclosure:

TABLE A
Light and heavy chain CDRs of an exemplary
anti-human PD-1 antibody

| | |
|---|---|
| LCDR1 | SEQ ID NO: 1 |
| LCDR2 | SEQ ID NO: 2 |
| LCDR3 | SEQ ID NO: 3 |
| HCDR1 | SEQ ID NO: 4 |
| HCDR2 | SEQ ID NO: 5 |
| HCDR3 | SEQ ID NO: 6 |

An example of anti-PD-1 antibody that binds to human PD-1 and can be used in the use, therapy, drug combination and kit of the present disclosure is described in WO2014206107. Human PD-1 mAbs that can be used as anti-PD-1 antibody in the use, therapy, drug combination and kit described herein include any one of the anti-PD-1 antibodies described in WO2014206107, including toripalimab (a humanized IgG4 mAb having the structure described in WHO Drug Information; 32(2):372-373 (2018) and comprising light and heavy chain amino acid sequences set forth in SEQ ID NOs: 9 and 10). In a preferred embodiment, the anti-PD-1 antibody that can be used for any one of the use, therapy, drug combination and kit of the present disclosure is selected from humanized antibodies 38, 39, 41 and 48 described in WO2014206107. In a particularly preferred embodiment, the anti-PD-1 antibody that can be used for any one of the use, therapy, drug combination and kit of the present disclosure is toripalimab.

The anti-PD-1 antibody that can be used for any one of the use, therapy, drug combination and kit of the present disclosure also include Nivolumab and Pembrolizumab that have been approved by FDA.

In some embodiments, the anti-PD-1 antibody that can be used for any one of the use, therapy, drug combination and kit of the present disclosure also include an anti-PD-L1 monoclonal antibody that specifically binds to PD-L1 to block the binding of PD-L1 to PD-1, such as nivolumab, pembrolizumab, toripalimab, Sintilimab, Camrelizumab, Tislelizumab and Cemiplimab.

“PD-L1” expression or “PD-L2” expression described herein refers to any detectable expression level of a specific PD-L protein on the surface of a cell or a specific PD-L mRNA within a cell or tissue. PD-L protein expression can be detected in IHC analysis of tumor tissue sections or by flow cytometry using diagnostic PD-L antibodies. In one embodiment, PD-L protein expression of tumor cells can be detected by PET imaging using a binding agent that specifically binds to a desired PD-L target (such as PD-L1 or PD-L2).

Methods for quantifying PD-L1 protein expression in IHC analysis of tumor tissue sections are found in, but are not limited to, Thompson, R. H. et al, PNAS 101(49):17174-17179 (2004); Taube, J. M. et al, Sci Transl Med 4, 127ra37 (2012); and Toplian, S. L. et al, New Eng. J. Med. 366(26): 2443-2454 (2012), and the like.

In one method, a simple binary endpoint of positive or negative PD-L1 expression is adopted, where the positive expression is defined by the percentage of tumor cells showing histological evidence of cell surface membrane staining. The case where tumor cells on a tumor tissue section account for at least 1% of the total tumor cells is defined as PD-L1 positive.

In another method, PD-L1 expression in the tumor tissue section is quantified in tumor cells as well as in infiltrating immune cells. The percentage of tumor cells and infiltrating immune cells exhibiting membrane staining are quantified individually as <1%, 1% to 50%, and subsequent 50% to 100%. For tumor cells, the PD-L1 expression is counted as negative if the score is <1%, and positive if the score is ≥1%.

In some embodiments, the expression level of PD-L1 by malignant cells and/or by infiltrating immune cells within the tumor is determined to be “overexpressed” or “elevated” based on comparison with the expression level of PD-L1 by an appropriate control. For example, the protein or mRNA expression level of control PD-L1 can be a quantified level in non-malignant cells of the same type or in sections from matched normal tissue.

### Paclitaxel

Paclitaxel is a natural secondary metabolite separated and purified from the bark of the gymnosperm yew, and has good anti-tumor effect through clinical verification. The chemical name of paclitaxel is 5β, 20-epoxy-1,2α,4,7β,10β,13α-hexahydroxytax-11-en-9-one-4,10-diacetate 2-benzoate 13-ester with (2R,3S)—N-benzoyl-3-phenylisoserine, which has a structure shown in the formula below.

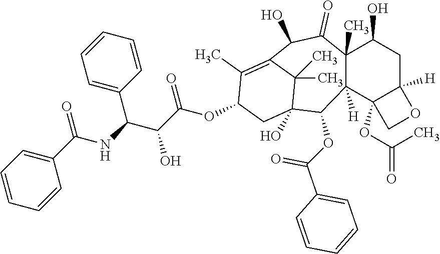 [SMILES: CC(=O)O[C@@]12CO[C@@H]1C[C@H](O)[C@@]1(C)C(=O)[C@H](O)C3=C(C)[C@@H](OC(=O)[C@H](O)[C@@H](NC(=O)c4ccccc4)c4ccccc4)C[C@](O)([C@@H](OC(=O)c4ccccc4)[C@H]21)C3(C)C]

In some embodiments of the present disclosure, paclitaxel may also refer to a composition comprising a therapeutically effective amount of a compound represented by the structural formula, a pharmaceutically acceptable salt thereof, and a pharmaceutically acceptable excipient. In some embodiments, the paclitaxel of the present disclosure is albumin-bound paclitaxel.

### Cisplatin

Cisplatin, the first metal complex with anti-cancer activity, is first discovered in 1965 by American scientists B. Rosenborg et al. Cisplatin, also known as cis-dichlorodiammineplatinum, is a platinum-containing anti-cancer drug in a form of orange yellow or yellow crystalline powder, which is slightly soluble in water and readily soluble in dimethylformamide and can be gradually converted into trans and hydrolyzed in aqueous solution. Cisplatin can clinically show efficacy on various solid tumors. Cisplatin is a compound having a structure shown below:

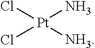

In some embodiments of the present disclosure, cisplatin may also refer to a composition comprising a therapeutically effective amount of a compound represented by the structural formula, a pharmaceutically acceptable salt thereof, and a pharmaceutically acceptable excipient. In some embodiments, cisplatin refers to a composition comprising (cis)diamminedichloroplatinum, sodium chloride and polyethylene glycol 400.

### Drug Combination

The present disclosure provides a drug combination comprising the anti-PD-1 antibody, the paclitaxel and the cisplatin of the present disclosure. In the drug combination, the anti-PD-1 antibody, the paclitaxel and the cisplatin may be provided in a mixture of the three (i.e., in a form of a pharmaceutical composition), or in a mixture of any two and another separate formulation, or each in separate formulation. In some embodiments, the drug combination comprises an administration dose for three weeks, including 1 dose of the anti-PD-1 antibody disclosed herein, 1 dose of paclitaxel and 1 dose of cisplatin. When present as separate formulations, each formulation may also comprise a pharmaceutically acceptable carrier in addition to the active ingredient. In one embodiment, the anti-PD-1 antibody or the antigen-binding fragment thereof, the paclitaxel and the cisplatin are in a liquid dosage form.

In some embodiments, the anti-PD-1 antibody of the present disclosure may be preferably as described in any one of the embodiments herein, more preferably an antibody comprising light chain CDRs with amino acid sequences set forth in SEQ ID NOs: 1, 2 and 3 and heavy chain CDRs with amino acid sequences set forth in SEQ ID NOs: 4, 5 and 6, even more preferably a monoclonal antibody comprising a light chain variable region set forth in SEQ ID NO: 7 and a heavy chain variable region set forth in SEQ ID NO: 8, still more preferably a monoclonal antibody comprising a light chain set forth in SEQ ID NO: 9 and a heavy chain set forth in SEQ ID NO: 10, yet more preferably the humanized antibodies 38, 39, 41 and 48 described in Patent No. WO2014206107, and most preferably toripalimab.

As used herein, “pharmaceutically acceptable carrier” includes any and all solvents, dispersion media, coatings, antibacterial and antifungal agents, isotonic and absorption delaying agents, and the like that are physiologically compatible. In one embodiment, the carrier suitable for the composition comprising the anti-PD-1 antibody is suitable for intravenous, intramuscular, subcutaneous, parenteral, spinal, or epidermal administration, such as by injection or infusion, while the carrier suitable for the composition comprising an additional anti-cancer agent is suitable for parenteral administration, such as oral administration. The drug combination of the present disclosure can contain one or more pharmaceutically acceptable salts, antioxidants, water, non-aqueous carriers, and/or adjuvants such as preserving agent, wetting agent, emulsifying agent, and dispersing agent.

The drug combination of the present disclosure may also comprise one or more additional therapeutic agents. The additional therapeutic agent can be, for example, a chemotherapeutic agent, a biotherapeutic agent, an immunogenic agent (e.g., an attenuated cancerous cell, a tumor antigen, an antigen-presenting cell (such as a dendritic cell pulsed with a tumor-derived antigen or nucleic acid), an immunostimulatory cytokine (e.g., IL-2, IFN-tumor, GM-CSF), and a cell transfected with a gene encoding an immunostimulatory cytokine (such as, but not limited to, GM-CSF)).

In one embodiment, each drug combination comprises a dose of the drug corresponding to the amount required for 1 treatment cycle.

### Dosage and Dosing Regimen

The choice of a dosing regimen (also referred to herein as an administration regimen) for the drug combination of the present disclosure depends on several factors, including the rate of solid serum or tissue turnover, the level of symptoms, the overall immunogenicity, and the degree of accessibility of the target cells, tissues or organs of the treated individual. In one embodiment, the dosing regimen maximizes the amount of each therapeutic agent delivered to the patient, consistent with acceptable level of side effects. Thus, the dosage and frequency of administration of each of the biotherapeutic and chemotherapeutic agents will depend, in part, on the particular therapeutic agent, the severity of cancer being treated, and the characteristics of patients. Guidance in selecting appropriate doses of antibodies, cytokines and small molecules may be obtained. Determination of an appropriate dosage regimen may be made by a clinician, for example, with reference to parameters or factors known or suspected to affect treatment or expected to affect treatment in the art, and will depend on, for example, the clinical history of the patients (e.g., previous treatments), the type and stage of cancer being treated, and biomarkers responsive to one or more therapeutic agents in the combination therapy.

Each therapeutic agent of the drug combination of the present disclosure may be administered simultaneously (i.e., in the same pharmaceutical composition), concurrently (i.e., in separate pharmaceutical formulations, one after the other in any order), or sequentially in any order. Sequential administration is particularly useful where the therapeutic agents in the drug combination can be in different dosage forms (one drug is a tablet or capsule and the other drug is a sterile liquid formulation) and/or on different dosing schedules (e.g., the chemotherapeutic agent is administered at least daily and the biotherapeutic agent is administered less frequently (e.g., once every week, once every two weeks or once every three weeks)).

In some embodiments, the therapeutic agent in at least one of the drug combinations is administered using the same dosage regimen (therapeutic dose, frequency and duration) that is generally used when the agent is used for treating the same tumor as a monotherapy. In other embodiments, the patient receives at least one of the therapeutic agents in the combination therapy at a lower total amount than that when the agent is used as a monotherapy, e.g., a lower dose, a lower dosing frequency, and/or a shorter duration of treatment.

Each therapeutic agent in the drug combination of the present disclosure can be administered orally or parenterally, including intravenous, intramuscular, intraperitoneal, subcutaneous, rectal, topical and transdermal routes of administration.

The anti-PD-1 antibody or the antigen-binding fragment thereof described herein can be administered by continuous infusion or by intermittent dosing. The single dose may be in a range of about 0.01 mg/kg to about 20 mg/kg or about 0.1 mg/kg to about 10 mg/kg body weight, or in a fixed dose of about 120 mg to about 480 mg. For example, the dose may be about 0.1 mg/kg, about 0.3 mg/kg, about 1 mg/kg, about 2 mg/kg, about 3 mg/kg, about 4 mg/kg, about 5 mg/kg, about 6 mg/kg, about 7 mg/kg, about 8 mg/kg, about 9 mg/kg or about 10 mg/kg body weight, or may be administered at a fixed dose of about 120 mg, 240 mg, 360 mg or 480 mg. Dosing regimens are generally designed to achieve an exposure that results in sustained receptor occupancy (RO) based on the typical pharmacokinetics of Ab. A representative dosing regimen may be about once a week, about once every two weeks, about once every three weeks, about once every four weeks, about once a month, or longer. In some embodiments, the anti-PD-1 antibody is administered to the individual about once every three weeks.

In some embodiments, the anti-PD-1 antibody described herein is toripalimab at a single dose selected from about 1 mg/kg to about 5 mg/kg body weight. In some embodiments, toripalimab is administered intravenously at a single dose selected from a dose of about 1 mg/kg, 2 mg/kg, 3 mg/kg, 3 mg/kg, 4 mg/kg and 5 mg/kg body weight, and a fixed dose of about 120 mg, 240 mg and 360 mg. In some preferred embodiments, toripalimab is administered as a liquid drug at a selected dose administered by intravenous infusion over a period of 30-60 min. In some embodiments, toripalimab is administered at about 3 mg/kg or a fixed dose of about 240 mg by intravenous infusion over a period of 30 min once every three weeks (Q3W). In some embodiments, toripalimab is administered at about 4.5 mg/kg or a fixed dose of about 360 mg by intravenous infusion over a period of 30 min once every three weeks (Q3W).

In some embodiments, paclitaxel described herein is continuously administered at an approved or recommended dose until a clinical effect is observed or until unacceptable toxicity or disease progression occurs. In some embodiments, paclitaxel described herein is administered at a single dose selected from about 130 mg/m² to about 230 mg/m² body surface area. In some embodiments, paclitaxel is administered at a single dose selected from about 130 mg/m², 145 mg/m², 160 mg/m², 175 mg/m², 190 mg/m², 205 mg/m² or 230 mg/m² body surface area. A representative dosing regimen may be about once a week, about once every two weeks, about once every three weeks, about once every four weeks or about once a month. In some embodiments, paclitaxel is administered to the individual once every three weeks. In some embodiments, paclitaxel is administered to the individual twice every three weeks. In some embodiments, paclitaxel is administered at about 175 mg/m² body surface area once every three weeks (Q3W).

As used herein, the body surface area (BSA) is defined by the DuBois formula: BSA (m²)=0.20247×height (m)^{0.725}×weight (kg)^{0.425}.

In some embodiments, cisplatin described herein is continuously administered at an approved or recommended dose until a clinical effect is observed or until unacceptable toxicity or disease progression occurs. In some embodiments, cisplatin described herein is administered at a single dose selected from about 60 mg/m² to about 90 mg/m² body surface area. In some embodiments, cisplatin described herein is administered at a single dose selected from about 60 mg/m², 65 mg/m², 70 mg/m², 75 mg/m², 80 mg/m², 85 mg/m² or 90 mg/m² body surface area. A representative dosing regimen may be about once a week, about once every two weeks, about once every three weeks, about once every four weeks or about once a month. In some embodiments, cisplatin is administered to the individual once every three weeks. In some embodiments, cisplatin is administered at about 75 mg/m² body surface area once every three weeks (Q3W).

In some embodiments, toripalimab is administered at a fixed dose of about 240 mg, Q3W, paclitaxel is administered at about 175 mg/m² body surface area, Q3W, and cisplatin is administered at about 75 mg/m² body surface area, Q3W.

In some embodiments, paclitaxel may be administered before or after toripalimab administration and cisplatin may be administered before or after toripalimab administration on the day of toripalimab administration.

The duration of treatment for the anti-PD-1 antibody or the antigen-binding fragment thereof, paclitaxel and cisplatin of the present disclosure may be the same or different, and may be one week, two weeks, three weeks, one month, two months, three months, four months, five months, half a year or longer. In one embodiment, the durations of treatment cycles may be the same or different, and the intervals between the treatment cycles may be the same or different. In some embodiments, the treatment cycle is three weeks. In some embodiments, in one treatment cycle, toripalimab is administered at a fixed dose of about 240 mg once every three weeks, paclitaxel is administered at about 175 mg/m² body surface area once every three weeks, and cisplatin is administered at about 75 mg/m² body surface area once every three weeks. The treatment cycles of the three are 4-6 weeks.

### Treatment Method and Usage

The present disclosure provides use of a combination of the anti-PD-1 antibody or the antigen-binding fragment thereof described herein and a chemotherapeutic agent in the preparation of a medicament for treating esophageal cancer. In one embodiment, the medicament is as described in any one of the embodiments herein.

The present disclosure further provides a method for preventing or treating esophageal cancer, comprising: administering to an individual in need an effective amount of the anti-PD-1 antibody or the antigen-binding fragment thereof described herein in combination with a chemotherapeutic agent, or the drug combination described herein. The effective amount includes a prophylactically effective amount and a therapeutically effective amount. In preferred embodiments, the dosing regimen of the prevention or treatment method is as described in any one of the embodiments herein.

The present disclosure provides a drug combination of the anti-PD-1 antibody or the antigen-binding fragment thereof described herein and a chemotherapeutic agent for preventing or treating esophageal cancer.

The esophageal cancer described herein can be as described in any one of the embodiments above; the esophageal cancer described herein is preferably esophageal squamous cell carcinoma, more preferably advanced or metastatic esophageal squamous cell carcinoma, and even more preferably advanced or metastatic esophageal squamous cell carcinoma without treatment with systemic chemotherapy.

In one embodiment, the method, the use, the anti-PD-1 antibody and the drug combination according to any one of the embodiments herein are particularly suitable for esophageal cancer with a gene amplification of chromosome 11q13 region.

In one embodiment, the method, the use and the drug combination according to any one of the embodiments herein are particularly suitable for esophageal cancer with a PD-L1 expression ≥1% in immunohistochemical staining analysis of a tumor tissue section, and preferably, esophageal cancer with a PD-L1 expression ≥10% in immunohistochemical staining analysis of tumor tissue section.

In one embodiment, the method, the use, the anti-PD-1 antibody and the drug combination according to any one of the embodiments herein are particularly suitable for esophageal cancer with a tumor mutation burden (TMB) less than 8 mutations/million base pairs, and preferably, esophageal cancer with a TMB less than 6 mutations/million base pairs.

The anti-PD-1 antibody for esophageal cancer may be preferably as described in any one of the embodiments herein, more preferably an antibody comprising light chain CDRs with amino acid sequences set forth in SEQ ID NOs: 1, 2 and 3 and heavy chain CDRs with amino acid sequences set forth in SEQ ID NOs: 4, 5 and 6, even more preferably a monoclonal antibody comprising a light chain variable region set forth in SEQ ID NO: 7 and a heavy chain variable region set forth in SEQ ID NO: 8, still more preferably a monoclonal antibody comprising a light chain set forth in SEQ ID NO: 9 and a heavy chain set forth in SEQ ID NO: 10, yet more preferably the humanized antibodies 38, 39, 41 and 48 described in Patent No. WO2014206107, and most preferably toripalimab.

The chemotherapeutic agent for esophageal cancer may be preferably as described in any one of the embodiments herein, and more preferably cisplatin and paclitaxel.

In some embodiments, the present disclosure provides a method for treating esophageal squamous cell carcinoma, comprising: administering to a patient with esophageal squamous cell carcinoma a therapeutically effective amount of toripalimab, cisplatin and paclitaxel, and the esophageal squamous cell carcinoma has a gene amplification of chromosome 11q13 region; preferably, the patient is positive for PD-L1 expression; preferably, the esophageal squamous cell carcinoma has a TMB less than 6 mutations/million base pairs. In one embodiment, the dosing regimen of the treatment method is as described in any one of the embodiments herein.

In more preferred embodiments, the present disclosure provides use of the anti-PD-1 antibody or the antigen-binding fragment thereof, cisplatin and paclitaxel in the preparation of a medicament for preventing or treating esophageal squamous cell carcinoma. In one embodiment, the esophageal squamous cell carcinoma has a gene amplification of chromosome 11q13 region; preferably, the patient is positive for PD-L1 expression; preferably, the esophageal squamous cell carcinoma has a TMB less than 6 mutations/million base pairs. In one embodiment, the dosing regimen of the treatment method is as described in any one of the embodiments herein.

### Method for Predicting Effect of Anti-PD-1 Antibody for Treating Cancer

The method for predicting the effect of the anti-PD-1 antibody or the antigen-binding fragment thereof, particularly toripalimab, in combination with a chemotherapeutic agent for treating esophageal cancer in an individual described herein comprise sequencing the individual prior to treatment. In some embodiments, the cancer is ESCC.

In one embodiment, the prediction method described herein is a gene test of an individual prior to treatment to assess the presence or absence of a gene amplification of the chromosome 11q13 region. In one embodiment, the gene amplification of the chromosome 11q13 region is absent in the individual. In another embodiment, the gene amplification of the chromosome 11q13 region is present in the individual.

The present disclosure further provides a method for predicting a therapeutic effect of an anti-PD-1 antibody or an antigen-binding fragment thereof in combination with a chemotherapeutic agent administered in a patient with tumor using genes of the chromosome 11q13 region. The presence of the gene amplification of the chromosome 11q13 region indicates that the patient with tumor is suitable for treatment with the combined use of the anti-PD-1 antibody or the antigen-binding fragment thereof and the chemotherapeutic agent.

In some embodiments, the present disclosure further provides use of a detection reagent of biomarkers in the preparation of a kit for predicting the effect of a combination of an anti-PD-1 antibody or an antigen-binding fragment thereof and a chemotherapeutic agent for treating esophageal cancer. Such reagents include, for example, those for detecting the presence or absence of the gene amplification of the chromosome 11q13 region in genes of an individual.

The present disclosure further provides use of a reagent for detecting genes of the chromosome 11q13 region in the preparation of a kit for predicting the effect of a combination of an anti-PD-1 antibody or an antigen-binding fragment thereof and a chemotherapeutic agent for treating esophageal cancer. Such reagents include, but are not limited to, those conventionally used in assays, including but not limited to, primers, probes, reagents required for PCR, and the like.

### Kit

The present disclosure further provides a kit comprising one or more single dosage units of the anti-PD-1 antibody or the antigen-binding fragment thereof according to any one of the embodiments herein, one or more single dosage units of paclitaxel and one or more single dosage units of cisplatin.

In some embodiments, the kit comprises one or more single dosage units of the drug combination according to any one of the embodiments herein.

In some embodiments, the kit comprises one or more sets of pharmaceutical formulations, each set of pharmaceutical formulations being the dosage of 3 weeks comprising 1 dose of toripalimab, 1 dose of paclitaxel and 1 dose of cisplatin. In one embodiment, toripalimab is a fixed dose of about 240 mg, the 1 dose of paclitaxel is a fixed dose of about 175 mg/m² body surface area, and the 1 dose of cisplatin is a fixed dose of about 75 mg/m² body surface area.

When present as separate formulations, each formulation may also comprise a pharmaceutically acceptable carrier in addition to the active ingredient.

In one embodiment, in the kit, one set of pharmaceutical formulations corresponds to 1 treatment cycle. In some embodiments, the kit may comprise 1-6 sets of pharmaceutical formulations for administration over 1-6 treatment cycles. In some embodiments, the kit may comprise 4-6 sets of pharmaceutical formulations for administration over 4-6 treatment cycles.

The kit disclosed herein can be used for treating esophageal cancer.

### Abbreviation

The following abbreviations are used throughout the description and examples of the present disclosure:
BID Two doses a day
CDR Complementarity determining region
DFS Disease-free survival
FR Framework region
IgG Immunoglobulin G
IHC Immunohistochemistry
OR Overall response
ORR Objective response rate
DCR Disease control rate
OS Overall survival
mOS Mean overall survival
PD Progressive disease
PFS Progression-free survival
mPFS Mean progression-free survival
PR Partial response
CR Complete response
SD Stable disease
DLT Dose-limiting toxicity
MTD Maximum tolerated dose
Q2W One dose every 2 weeks
QD One dose everyday
CSD Chronic sun-damaged
non-CSD Non-chronic sun-damaged
IRC Independent review committee
AE Adverse event
TRAE Treatment-related adverse event
SAE Serious adverse event
RO Receptor occupancy
UC Urothelial carcinoma
RCC Renal cell carcinoma
MM Metastatic melanoma
RECIST Response Evaluation Criteria in Solid Tumor
irRECIST Immune-Related Response Evaluation Criteria in Solid Tumor
DOR Duration of response
TTR Time to response
BIRC Blinded individual review committee
NE Not evaluable

The present disclosure is further illustrated by the following examples, which should not be construed as limiting the present disclosure. The contents of all references cited throughout the present application are explicitly incorporated herein by reference.

### EXAMPLES

Example 1: Clinical study of anti-PD-1 antibody in combination with first-line chemotherapy for treating advanced or metastatic esophageal squamous cell carcinoma that is not previously treated systemic chemotherapy

### 1 Study Design

This is a randomized, double-blind, placebo-controlled, multicenter, phase III clinical study evaluating the efficacy and safety of toripalimab (JS001) or placebo in combination with first-line chemotherapy in patients with advanced or metastatic esophageal squamous cell carcinoma that are not previously treated with systemic chemotherapy and exploring the population with optimal biomarker prediction.

### 1.1 Primary Inclusion Criteria:

Eligible subjects shall meet the following criteria: (1) aged between 18 and 75 years; (2) histologically or pathologically confirmed metastatic or relapsed esophageal squamous cell carcinoma; (3) previously not treated for relapsed or metastatic ESCC; (4) at least one measurable lesion (according to RECIST 1.1); (5) an ECOG score of 0 or 1; (6) normal organ functions.

### 1.2 Study Design:

Approximately 514 patients with advanced or metastatic esophageal squamous cell carcinoma were randomized into two groups in a ratio of 1:1, with the treatment group receiving toripalimab in combination with first-line chemotherapy and the control group receiving placebo in combination with first-line chemotherapy. The patients were stratified by ECOG score (0 vs 1) and previous radiotherapy (present vs absence).

After randomization, the patients were subject to combination chemotherapy, receiving toripalimab or placebo+first-line chemotherapy for up to 6 cycles. After that, the patients were subject to maintenance therapy, continuously receiving toripalimab or placebo treatment until disease progression, intolerable toxicity, withdrawal from the study or up to 2 years.

Toripalimab 240 mg/placebo was administered by intravenous infusion once every three weeks (Q3W) in treatment cycles of 21 days. Treatment continued until the patients met the discontinuation criteria, i.e., recorded disease progression, unacceptable AE, investigator-assessed unsuitability for continued treatment, withdrawal of informed consent, 2 years of JS001 treatment or other reasons as specified in the protocol.

Paclitaxel+cisplatin treatment (TP) during the combination treatment period: paclitaxel 175 mg/m² and cisplatin at 75 mg/m² were administered by intravenous drip infusion on day 1 of every 21-day treatment cycle, i.e., once every three weeks (Q3W).

### 1.3 Subjects:

By March, 2021, a total of about 514 patients with advanced or metastatic esophageal squamous cell carcinoma were enrolled, with about 257 in the treatment group and about 257 in the control group. Demographics of the enrolled subjects are shown in Table 1.

TABLE 1
Demographic data of the enrolled subjects

| | JS001 + TP | | Placebo + TP | | Total | |
|---|---|---|---|---|---|---|
| Number of subjects (%) | (N = 257) | | (N = 257) | | (N = 514) | |
| Age (years) | 63.0 | (20-75) | 62.0 | (40-74) | 62.5 | (20-75) |
| Age | | | | | | |
| <65 years | 156 | (60.7) | 163 | (63.4) | 319 | (62.1) |
| ≥65 years | 101 | (39.3) | 94 | (36.6) | 195 | (37.9) |
| Sex | | | | | | |
| Male | 217 | (84.4) | 220 | (85.6) | 437 | (85.0) |
| Female | 40 | (15.6) | 37 | (14.4) | 77 | (15.0) |
| Ethnicity | | | | | | |
| Han | 245 | (95.3) | 243 | (94.6) | 488 | (94.9) |
| Others | 12 | (4.7) | 14 | (5.4) | 26 | (5.1) |
| BMI (kg/m²) | 21.4 | (14.3-33.4) | 21.3 | (14.5-33.7) | 21.3 | (14.3-33.7) |
| Stage of disease | | | | | | |
| Distant metastasis | 206 | (80.2) | 198 | (77.0) | 404 | (78.6) |
| Local | 50 | (19.5) | 59 | (23.0) | 109 | (21.2) |
| relapsed/unresectable | | | | | | |
| NA | 1 | (0.4) | 0 | | 1 | (0.2) |
| ECOG score - CRF | | | | | | |
| 0 | 66 | (25.7) | 65 | (25.3) | 131 | (25.5) |
| 1 | 191 | (74.3) | 192 | (74.7) | 383 | (74.5) |
| Previous radiotherapy - CRF | | | | | | |
| Yes | 35 | (13.6) | 34 | (13.2) | 69 | (13.4) |
| No | 222 | (86.4) | 223 | (86.8) | 445 | (86.6) |
| PD-L1 expression | | | | | | |
| CPS < 1 | 43 | (16.7) | 44 | (17.1) | 87 | (16.9) |
| CPS ≥ 1 | 201 | (78.2) | 200 | (77.8) | 401 | (78.0) |
| CPS < 10 | 129 | (50.2) | 147 | (57.2) | 276 | (53.7) |
| CPS ≥ 10 | 115 | (44.7) | 97 | (37.7) | 212 | (41.2) |
| NA | 13 | (5.1) | 13 | (5.1) | 26 | (5.1) |

Note:
BMI (kg/m²) = weight (kg)/height (m²); TP scheme: paclitaxel + cisplatin first-line chemotherapy; CPS: combined positive score. CPS is defined as: the fraction of tumor cells with any intensity of membrane staining and lymphocytes/macrophages directly associated with tumor cells with membrane/cytoplasmic staining relative to total tumor cells (at least one hundred), excluding staining of total necrotic cells, interstitial cells, carcinoma in situ, other immune cells (including but not limited to neutrophils, eosinophils and plasma cells) and the like.

### 1.4 Investigational Product:

JS001: toripalimab (WO2014206107, Junshi Biosciences).

### 1.5 Treatment Duration:

Toripalimab injection 240 mg/placebo was administered by intravenous infusion Q3W in treatment cycles of 21 days. Treatment continued until the patient met the discontinuation criteria, i.e., recorded disease progression, unacceptable AEs, investigator-assessed unsuitability for continued treatment, withdrawal of informed consent, 2 years of JS001 treatment or other reasons as specified in the protocol.

The patients received JS001/placebo+first-line chemotherapy during the combination chemotherapy period (up to 6 cycles). After that, the patients were subject to maintenance monotherapy, continuously receiving JS001/placebo treatment until disease progression, intolerable toxicity, withdrawal from the study or up to 2 years.

### 1.6 Statistics:

### Analysis Set

Intent-to-Treat (ITT) set: including all randomized patients during the primary study period. This analysis set was used as the primary analysis set for efficacy analysis. Per protocol set (PPS): including all ITT population having valid baseline information without major violation to the protocol that may affect the efficacy analysis. The PPS population was determined based on actual protocol violations before database lock of the study. The PPS population was used for sensitivity analysis of the primary efficacy endpoints as well as part of the secondary efficacy endpoints. Safety analysis set (SS): including all patients who received at least one dose of the study drug (JS001/placebo/chemotherapeutic agent).

### Efficacy Analysis

Unless otherwise specified, the efficacy analysis was based on the ITT set by treatment.

The progression-free survival rate and median progression-free survival of the JS001+first-line treatment group and the placebo+first-line treatment group at different time points after the start of treatment were estimated by Kaplan-Meier method, and a Kaplan-Meier curve was plotted; the 95% confidence interval for progression-free survival rate at different time points (6 months and 1 year) was estimated using the Greenwood formula; the 95% confidence interval for median progression-free survival was estimated using the Brookmeyer-Crowley method where the log-log function conversion was used to reach normal approximation. Statistical test was performed on the inter-group difference by a stratified log-rank method for the total population. Statistical test was performed on the inter-group difference by a non-stratified log-rank method for the subpopulations. A stratified COX proportional hazards model was used to estimate the inter-group hazard ratio (HR) and the corresponding 95% confidence interval, and the tied events were processed by an exact method.

For analysis of other efficacy endpoints OS, DOR, DCR and ORR, the method described above as applied to the primary efficacy endpoint PFS was used. For ORR and DCR, the percentage of patients in each group was calculated, and their 95% confidence intervals were calculated using the Clopper-Pearson method. The nominal P values for inter-group comparison were calculated using the Cochran-Mantel-Haenszel method considering randomization stratification factors that were the same as the primary efficacy analysis, and the 95% CI for the inter-group differences was estimated.

### 2 Results

809 patients were screened in this study and 514 patients were randomized (257 in the treatment group and 257 in the control group). All patients were included in the ITT population as well as the SS population.

The demographics and baseline disease characteristics were substantially similar for two groups. Most enrolled patients were less than 65 years old (62.1%) and most were male (85.0%) with ECOG score of 1 (74.5%), previous radiotherapy (86.6%) and PD-L1 expression CPS ≥1% (78.0%). In summary, in the ITT population, the baseline characteristics of patients were similar for the treatment group and the control group with no significant difference.

### Efficacy Results

One of the primary endpoints of the study was OS (ITT). As of Mar. 22, 2021, the median follow-up time was 7.343 months. The interim analysis results showed that the primary endpoint reached the preset superiority margin, and for all subpopulations, the analysis results of the treatment group were superior to those of the control group, including the PD-L1 expression subpopulation. For the other primary endpoint PFS (ITT) of this study, the BICR assessment reached the preset superiority margin and was consistent with the investigator-assessed PFS benefit. For all subpopulations, the analysis results of the treatment group were superior to those of the control group. In terms of other efficacy endpoints (ORR, DCR, DOR, DCR, etc.), the treatment group was superior.

The results for the primary and secondary efficacy endpoints in the ITT set are as follows:

### 2.1 Overall Survival (OS; Primary Endpoint)

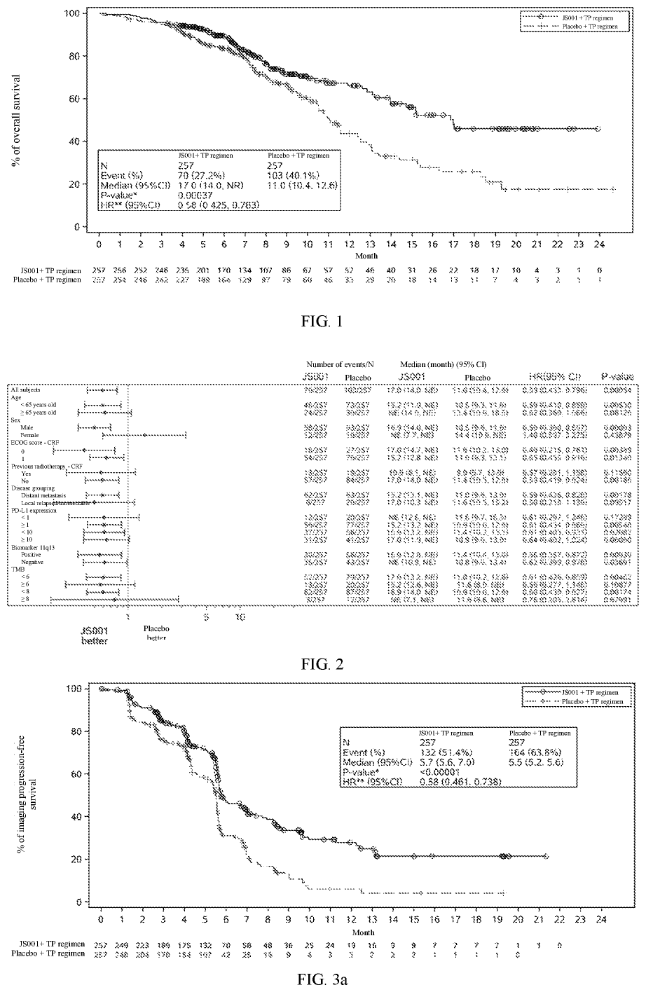

As of Mar. 22, 2021, the median follow-up time was 7.343 months, a total of 173 deaths were reported, 70 (27.2%) in the treatment group and 103 (40.1%) in the control group. As shown in FIG. 1, the median OS was 17.0 months (95% CI: 14.0, NR) for the treatment group and 11.0 months (95% CI: 10.4, 12.6) for the control group in the ITT population (HR=0.58; 95% CI: 0.425, 0.783; P=0.00037). Toripalimab in combination with chemotherapy significantly prolonged the median OS by 6 months and reduced the death hazard by 42% versus chemotherapy in patients with esophageal cancer, reaching the primary efficacy endpoint margin as preset for interim analysis. The 1-year PFS rate was 66.0% (57.5, 73.2) for the treatment group and 43.7% (34.4, 52.6) for the control group, and the 1-year OS rate of the treatment group was significantly improved compared with that of the control group, demonstrating a stable benefit trend.

Meanwhile, the OS subpopulation analysis showed that the results of ECOG score (0 vs 1), previous radiotherapy, disease stage, PD-L1 expression (CPS <1 vs CPS ≥1, CPS <10 vs CPS ≥1), biomarker 11q13 positive/negative subpopulations, TMB (TMB <6 vs TMB ≥6, TMB <8 vs TMB ≥8) supported the superiority in OS of the treatment group to the control group (see FIG. 2).

### 2.2 Progression-Free Survival (PFS, Primary Endpoint)

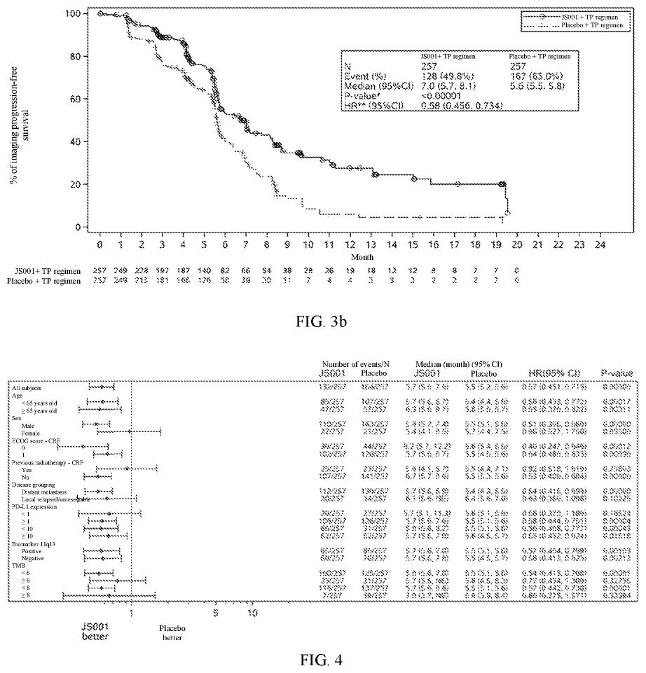

As shown in FIG. 3a, the median PFS was 5.7 months (95% CI: 5.6, 7.0) for the treatment group and 5.5 months (95% CI: 5.2, 5.6) for the control group in the ITT population (HR 0.58; 95% CI: 0.461, 0.738; P<0.00001) in patients with esophageal cancer as assessed by BIRC based on RECIST 1.1. Toripalimab in combination with chemotherapy significantly improved the progression-free survival and reduced the progression or death hazard by 42% versus chemotherapy, reaching the primary efficacy endpoint margin as preset for interim analysis. The 1-year PFS rate was 27.8% (20.4, 35.8) for the treatment group and 6.1% (2.2, 12.6) for the control group, and the 1-year PFS rate of the treatment group was significantly improved compared with that of the control group, demonstrating a stable benefit trend.

As shown in FIG. 3b, the median PFS was 7.0 months (95% CI: 5.7, 8.1) for the treatment group and 5.6 months (95% CI: 5.5, 5.8) for the control group in the ITT population (HR=0.58; 95% CI: 0.456, 0.734; P<0.00001) in patients with esophageal cancer as assessed by the investigator based on RECIST 1.1. Toripalimab in combination with chemotherapy significantly improved the progression-free survival and reduced the progression or death hazard by 42% versus chemotherapy. The 1-year OS rate was 27.6% (19.9, 35.8) for the treatment group and 6.1% (2.4, 12.1) for the control group, and the 1-year PFS rate of the treatment group was significantly improved compared with that of the control group, demonstrating a stable benefit trend.

Meanwhile, the PFS subpopulation analysis showed that the results of ECOG score (0 vs 1), previous radiotherapy, disease stage, PD-L1 expression (CPS <1 vs CPS ≥1, CPS <10 vs CPS ≥1), biomarker 11q13 positive/negative subpopulations, TMB (TMB <6 vs TMB ≥6, TMB <8 vs TMB ≥8) supported the superiority in PFS of the treatment group to the control group (see FIG. 4).

### 2.3 Objective Response Rate (ORR) and Disease Control Rate (DCR)

The ORR was 69.3% for the treatment group and 52.1% for the control group as assessed by BIRC according to RECIST v1.1. The treatment group showed a 17.2% increase in ORR versus the control group. The DCR was 89.1% for the treatment group and 82.1% for the control group, both being greater than 80%, indicating a well control of the tumor.

The ORR was 72.4% for the treatment group and 58.4% for the control group as assessed by the investigator according to RECIST v1.1. The treatment group showed a 14% increase in ORR versus the control group. The DCR was 92.6% for the treatment group and 87.2% for the control group, both being greater than 80%, indicating a well control of the tumor.

The results are shown in Table 2, and the two sets of data showed that the ORR of the treatment group was superior to that of the control group.

### 2.4 Duration of Response (DOR)

In 312 patients (178 in the treatment group and 134 in the control group) with BOR of CR or PR, the median DOR was 5.6 months for the treatment group and 4.2 months for the control group as assessed by BIRC according to RECIST 1.1 (HR 0.58; 95% CI: 0.412, 0.810), and the treatment group demonstrated a significantly prolonged DOR over the control group by 1.4 months. The 1-year DOR rate was 28.2% (17.9, 39.3) for the treatment group and 4.7% (0.5, 17.4) for the control group, and the 1-year DOR rate of the treatment group was significantly improved compared with that of the control group, demonstrating a stable benefit trend.

In patients with BOR of CR or PR, the duration of response was significantly prolonged in both treatment group and control group, as shown in Table 2.

TABLE 2
Investigator/BICR assessment according to RECIST 1.1 criteria (esophageal cancer subjects)

| | BICR-assessed | | | | Investigator-assessed | | | |
|---|---|---|---|---|---|---|---|---|
| | JS001 + TP | | Placebo + TP | | JS001 + TP | | Placebo + TP | |
| Number of subjects (%) | (N = 257) | | (N = 257) | | (N = 257) | | (N = 257) | |
| Best overall response (BOR) | | | | | | | | |
| Complete response (CR) | 30 | (11.7) | 18 | (7.0) | 13 | (5.1) | 8 | (3.1) |
| Partial response (PR) | 148 | (57.6) | 116 | (45.1) | 173 | (67.3) | 142 | (55.3) |
| Stable disease (SD) | 51 | (19.8) | 77 | (30.0) | 52 | (20.2) | 74 | (28.8) |
| Progressive disease (PD) | 18 | (7.0) | 35 | (13.6) | 10 | (3.9) | 24 | (9.3) |
| non-CR/non-PD | 1 | (0.4) | 2 | (0.8) | / | | / | |
| Not evaluable (NE) | 0 | | 0 | | 0 | | 0 | |
| Not applicable | 0 | | 0 | | / | | / | |
| Objective response rate | 178 (69.3), | | 134 (52.1), | | 186 (72.4), | | 150 (58.4), | |
| (ORR) | 95% CI(63.2, 74.8) | | 95% CI(45.8, 58.4) | | 95% CI(66.5, 77.7) | | 95% CI(52.1, 64.5) | |
| Inter-group difference (95% CI) | 17.2 (9.0, 25.4) | | | | 14.1 (6.0, 22.2) | | | |
| Inter-group comparison | P < 0.0001 | | | | P = 0.0007 | | | |
| Disease control rate (DCR) | 229 (89.1), | | 211 (82.1), | | 238 (92.6), | | 224 (87.2), | |
| | 95% CI(84.6, 92.6) | | 95% CI(76.9, 86.6) | | 95% CI(88.7, 95.5) | | 95% CI(82.4, 91.0) | |
| Inter-group difference (95% CI) | 7.1 (1.1, 13.1) | | | | 5.5 (0.3, 10.7) | | | |
| Inter-group comparison | P = 0.0206 | | | | P = 0.0371 | | | |
| Duration of response (DOR) | | | | | | | | |
| Median (month, 95% CI) | 5.6 | (4.4, 8.7) | 4.2 | (4.2, 4.4) | 4.2 | (3.1, 4.4) | 3.0 | (2.7, 3.9) |
| HR (95% CI), P value | 0.58 (0.412, 0.810), P = 0.00136 | | | | 0.50 (0.359, 0.684), P = 0.00001 | | | |
| 6-month DOR (%, 95% CI) | 47.4 | (37.9, 56.2) | 28.4 | (18.7, 38.9) | 55.6 | (46.2, 64.0) | 28.7 | (19.5, 38.5) |
| 12-month DOR (%, 95% CI) | 28.2 | (17.9, 39.3) | 4.7 | (0.5, 17.4) | 30.9 | (20.5, 41.9) | 3.8 | (0.4, 14.4) |

Note:
TP: paclitaxel + cisplatin first-line chemotherapy; the 95% confidence intervals for objective response rate and disease control rate were calculated for the two groups using an exact method (Clopper-Pearson).

### 2.5 Safety Results

All safety analyses were based on the SS population (514 patients), including 257 patients in the treatment group and 257 patients in the control group. Both groups were sufficiently exposed to JS001+chemotherapy treatment or placebo+chemotherapy treatment. The primary safety analysis results based on the SS population in the double-blind phase of the primary study are as follows:

The proportion of patients who experienced at least 1 TEAE was 99.2% for both groups; the proportion of TEAEs of grade 3 or higher as per CTCAE was 73.2% for the treatment group and 70.0% for the control group, and the combined use with JS001 did not increase the incidence of adverse events of grade 3 or higher on the basis of first-line chemotherapy. During the treatment period, the proportion of treatment-emerged SAEs was 36.2% and 28.8% for the treatment group and the control group, respectively, and the proportion of SAEs of grade 3 or higher as per CTCAE was 30.0% and 26.5% for the treatment group and the control group, respectively. The two groups showed similar proportions, and the treatment group demonstrated no increase in adverse event resulting in death.

95 (37%) and 68 (26.5%) patients reported immune-related adverse events (irAEs) in the treatment group and the control group, respectively, as assessed by the investigator in the blind state, most of grade 1-2; 17 (6.6%) and 4 (1.6%) subjects in the treatment group and the control group reported irAEs of grade 3 or higher, respectively; no irAE resulting in death was reported in the treatment groups. The incidence and severity of immune-related TEAEs were similar to other drugs of the kind (PD-1 blocking antibodies) as expected.

Common TEAEs included anemia (77.0% for treatment group vs 77.8% for control group), decreased neutrophil count (66.9% vs 54.1%), decreased leukocyte count (67.7% vs 53.7%), decreased platelet count (28.4% vs 16.7%), asthenia (41.6% vs 38.1%), alopecia (35.4% vs 40.5%), decreased appetite (39.3% vs 45.9%), nausea (42.8% vs 45.5%), vomiting (40.1% vs 36.6%), diarrhea (23.0% vs 14.0%), constipation (24.1% vs 21.4%), weight loss (28.8% vs 28.4%), etc., most of which were common related adverse events and demonstrated similar incidences between the treatment group and the control group, indicating no increase in chemotherapy-related toxicity by combined use with JS001 on the basis of chemotherapy.

Common SAEs included vomiting (8.9% vs 8.2%), dysphagia (1.2% vs 1.2%), infectious pneumonitis (5.1% vs 2.3%), decreased leukocyte count (2.3% vs 1.9%), decreased platelet count (1.6% vs 2.7%), decreased neutrophil count (1.6% vs 2.7%), anemia (1.2% vs 0.4%), bone marrow depression (1.2% vs 0.8%), asthenia (1.2% vs 0.4%), etc., suggesting a low overall incidence of JS001-related SAEs.

Safety result summary: in the SS population in the double-blind phase of the primary study, the proportions of TEAEs, TEAEs of grade ≥3 as per CTCAE, SAEs and SAEs of grade ≥3 as per CTCAE between the treatment group and the control group were similar, indicating no increase in chemotherapy-related toxicity by combined use with JS001 on the basis of chemotherapy. JS001-related adverse reactions were similar to those observed in previous studies, and no new safety signs were found.

### CONCLUSION

The interim analysis results showed that toripalimab in combination with chemotherapy significantly prolonged the OS and PFS and reduced the progression or death hazard versus the first-line chemotherapy in patients with esophageal cancer; among the secondary efficacy endpoints, ORR and DOR also demonstrated a significant increase in the toripalimab group. Meanwhile, the tolerability and safety in target indicated population are generally controllable, and no new safety signal was found.

1. A method of preventing or treating esophageal cancer, comprising administering to an individual in need thereof a therapeutically effective amount of the anti-PD-1 antibody or the antigen-binding fragment thereof in combination with a chemotherapeutic agent,
wherein the esophageal cancer is advanced or metastatic esophageal squamous cell carcinoma.

2. (canceled)

3. The method according to claim 1, wherein the esophageal cancer is esophageal cancer with a gene amplification of chromosome 11q13 region.

4. The method according to claim 1, wherein the esophageal cancer is esophageal cancer with a PD-L1 expression of <1%, a PD-L1 expression of ≥1%, a PD-L1 expression of <10% or a PD-L1 expression of ≥10% in immunohistochemical staining analysis of a tumor tissue section.

5. The method according to claim 1, wherein the esophageal cancer is esophageal cancer with a tumor mutation burden (TMB) of less than 8 mutations/million base pairs.

6. The method according to claim 1, wherein the anti-PD-1 antibody or the antigen-binding fragment thereof comprises a light chain complementarity determining region having amino acid sequences set forth in SEQ ID NOs: 1, 2 and 3, and a heavy chain complementarity determining region having amino acid sequences set forth in SEQ ID NOs: 4, 5 and 6.

7. The method according to claim 6, wherein the anti-PD-1 antibody or the antigen-binding fragment thereof comprises a light chain variable region having an amino acid sequence set forth in SEQ ID NO: 7 and a heavy chain variable region having an amino acid sequence set forth in SEQ ID NO: 8.

8. The method according to claim 7, wherein the anti-PD-1 antibody comprises a light chain having an amino acid sequence set forth in SEQ ID NO: 9 and a heavy chain having an amino acid sequence set forth in SEQ ID NO: 10.

9. The method according to claim 1, wherein the anti-PD-1 antibody is toripalimab.

10. The method according to claim 1, wherein the chemotherapeutic agent is selected from platinum and paclitaxel.

11. The method according to claim 1, wherein the combination is a combination of toripalimab, cisplatin and paclitaxel.

12. The method according to claim 1, wherein:
the anti-PD-1 antibody or the antigen-binding fragment thereof is administered at a single dose of about 0.1 mg/kg body weight to about 10.0 mg/kg body weight, or of about 0.1 mg/kg body weight, 0.3 mg/kg body weight, 1 mg/kg body weight, 2 mg/kg body weight, 3 mg/kg body weight, 5 mg/kg body weight or 10 mg/kg body weight, or selected from a fixed dose of about 120 mg to about 480 mg, or of a fixed dose of about 120 mg, 240 mg, 360 mg or 480 mg;
the paclitaxel is administered at a single dose of about 130 mg/m² body surface area to about 230 mg/m² body surface area, or of about 130 mg/m² body surface area, 145 mg/m² body surface area, 160 mg/m² body surface area, 175 mg/m² body surface area, 190 mg/m² body surface area, 205 mg/m² body surface area or 230 mg/m² body surface area; and
the cisplatin is administered at a single dose of about 60 mg/m² body surface area to about 90 mg/m² body surface area, or of about 60 mg/m² body surface area, 65 mg/m² body surface area, 70 mg/m² body surface area, 75 mg/m² body surface area, 80 mg/m² body surface area, 85 mg/m² body surface area or 90 mg/m² body surface area.

13. The method according to claim 12, wherein:
the anti-PD-1 antibody or the antigen-binding fragment thereof is administered at a frequency of about once every week, once every two weeks, once every three weeks, once every four weeks or once a month;
the paclitaxel is administered at a frequency of about once every week, twice every three weeks, once every two weeks, once every three weeks, once every four weeks or once a month; and
the cisplatin is administered at a frequency of about once every week, twice every three weeks, once every two weeks, once every three weeks, once every four weeks or once a month.

14. The method according to claim 13, wherein:
the anti-PD-1 antibody or the antigen-binding fragment thereof is administered at a fixed dose of 240 mg or 360 mg once every three weeks; the paclitaxel is administered at a single dose of about 175 mg/m² body surface area once every three weeks; and the cisplatin is administered at a single dose of about 75 mg/m² body surface area once every three weeks.

15. The method according to claim 13, wherein:
the anti-PD-1 antibody or the antigen-binding fragment thereof, the paclitaxel and the cisplatin are administered for one week, two weeks, three weeks, one month, two months, three months, four months, five months, half a year or longer; in one embodiment, the durations of treatment cycles are the same or different, and the intervals between treatment cycles are the same or different; and/or
the anti-PD-1 antibody or the antigen-binding fragment thereof, the paclitaxel and the cisplatin are administered parenterally, or, by intravenous infusion, in a liquid dosage form, or in an injection.

16. (canceled)

17. A pharmaceutical combination comprising an anti-PD-1 antibody or an antigen-binding fragment thereof as defined in claim 6, paclitaxel and cisplatin.

18. The pharmaceutical combination according to claim 17, wherein
(1) the amount of the anti-PD-1 antibody or the antigen-binding fragment thereof in the pharmaceutical combination is sufficient to provide about 0.1 mg/kg body weight to about 10.0 mg/kg body weight, or of about 0.1 mg/kg body weight, 0.3 mg/kg body weight, 1 mg/kg body weight, 2 mg/kg body weight, 3 mg/kg body weight, 5 mg/kg body weight or 10 mg/kg body weight, or selected from a fixed dose of about 120 mg to about 480 mg, or of a fixed dose of about 120 mg, 240 mg, 360 mg or 480 mg, or the anti-PD-1 antibody or the antigen-binding fragment thereof at a single dose;
the amount of the paclitaxel in the pharmaceutical combination is sufficient to provide about 130 mg/m² body surface area to about 230 mg/m² body surface area, or of about 130 mg/m² body surface area, 145 mg/m² body surface area, 160 mg/m² body surface area, 175 mg/m² body surface area, 190 mg/m² body surface area, 205 mg/m² body surface area or 230 mg/m² body surface area, or the paclitaxel at a single dose; and
the amount of the cisplatin in the pharmaceutical combination is sufficient to provide about 60 mg/m² body surface area to about 90 mg/m² body surface area, or of about 60 mg/m² body surface area, 65 mg/m² body surface area, 70 mg/m² body surface area, 75 mg/m² body surface area, 80 mg/m² body surface area, 85 mg/m² body surface area or 90 mg/m² body surface area, or the cisplatin at a single dose; or
(2) the anti-PD-1 antibody or the antigen-binding fragment thereof is administered at a frequency of about once every week, once every two weeks, once every three weeks, once every four weeks or once a month;
the paclitaxel is administered at a frequency of about once every week, twice every three weeks, once every two weeks, once every three weeks, once every four weeks or once a month; and
the cisplatin is administered at a frequency of about once every week, twice every three weeks, once every two weeks, once every three weeks, once every four weeks or once a month.

19. (canceled)

20. A kit comprising
one or more single dosage units of an anti-PD-1 antibody or an antigen-binding fragment thereof as defined in claim 6, one or more single dosage units of paclitaxel and one or more single dosage units of cisplatin;
wherein:
(1) the anti-PD-1 antibody or the antigen-binding fragment thereof in one or more single dosage units comprising the anti-PD-1 antibody or the antigen-binding fragment thereof at a dose of about 120 mg to about 480 mg, e.g., a dose of 120 mg, 240 mg, 360 mg or 480 mg;
the paclitaxel in one or more single dosage units comprising the paclitaxel at about 130 mg/m² body surface area to about 230 mg/m² body surface area, e.g., about 130 mg/m² body surface area, 145 mg/m² body surface area, 160 mg/m² body surface area, 175 mg/m² body surface area, 190 mg/m² body surface area, 205 mg/m² body surface area or 230 mg/m² body surface area; and
the cisplatin in one or more single dosage units comprising the cisplatin at about 60 mg/m² body surface area to about 90 mg/m² body surface area, e.g., about 60 mg/m² body surface area, 65 mg/m² body surface area, 70 mg/m² body surface area, 75 mg/m² body surface area, 80 mg/m² body surface area, 85 mg/m² body surface area or 90 mg/m² body surface area; or
(2) the anti-PD-1 antibody or the antigen-binding fragment thereof is administered at a frequency of about once every week, once every two weeks, once every three weeks, once every four weeks or once a month;
the paclitaxel is administered at a frequency of about once every week, twice every three weeks, once every two weeks, once every three weeks, once every four weeks or once a month; and
the cisplatin is administered at a frequency of about once every week, twice every three weeks, once every two weeks, once every three weeks, once every four weeks or once a month.

21. (canceled)

22. A kit comprising one or more single dosage units of the pharmaceutical combination according to claim 17.

23. The method according to claim 5, the esophageal cancer is esophageal cancer with a tumor mutation burden (TMB) of less than 6 mutations/million base pairs.

24. The method according to claim 10, wherein the chemotherapeutic agent is selected from cisplatin and paclitaxel.
